(19) United States
(12) Patent Application Publication
(10) Pub. No.: US 2011/0251097 A1
(43) Pub. Date: Oct. 13, 2011

(54) DIAGNOSTIC KIT OF COLON CANCER USING COLON CANCER RELATED MARKER AND DIAGNOSTIC METHOD THEREOF

(75) Inventors: Eun Young Song, Seoul (KR); Hee Gu Lee, Daejeon (KR); Young Il Yeom, Daejeon (KR); Jae Wha Kim, Daejeon (KR); Na Young Ji, Gyeonggi-do (KR); Kyung-Sook Chung, Daejeon (KR); Misun Won, Daejeon (KR); Seon-Young Kim, Daejeon (KR); Joo Heon Kim, Daejeon (KR); Young Ho Kim, Seoul (KR); Ho Kyung Chun, Seoul (KR)

(73) Assignee: KOREA RESEARCH INSTITUTE OF BIOSCIENCE AND BIOTECHNOLOGY, Daejeon (KR)

(21) Appl. No.: 13/126,500

(22) PCT Filed: Apr. 20, 2009
(86) PCT No.: PCT/KR2009/002039

§ 371 Date: Apr. 28, 2011

(30) Foreign Application Priority Data
Oct. 22, 2008 (KR) 10-2008-0103387

(51) Int. Cl. C40B 30/04; G01N 33/53; G01N 33/566; C12Q 1/68

(52) U.S. Cl. 506/9; 435/6.14; 435/6.12; 436/501; 435/7.92; 435/7.1

(57) ABSTRACT

The present invention relates to a composition for diagnosing colon cancer. The composition comprises at least one marker for measuring an mRNA or protein expression level of at least one gene specific for colon cancer. It can screen the genes which are overexpressed specifically only in colon cancer tissues or blood. The present invention can quantitatively analyze both the mRNA expression levels of the genes and the expression levels of the proteins encoded by the gene at the same time, thereby diagnosing colon caner of an early stage with a high level of reliability.

### TECHNICAL FIELD

The present invention relates to a diagnostic kit of colon cancer using a colon cancer-related marker and a method of yielding information necessary for the diagnosis of colon cancer. More particularly, the present invention relates to a diagnostic composition for colon cancer, comprising at least one marker for measuring an mRNA or protein expression level of at least one gene specific for colon cancer, and a method of yielding information necessary for the diagnosis of colon cancer using the same.

### BACKGROUND ART

The large intestine is the last part of the digestive system in the body in which the food ingested through the mouth is digested and absorbed and even excess food is stayed. The main function of the large intestine is to transport waste out of the body and to absorb water from the waste before it leaves. In addition, the large intestine houses over 700 species of bacteria that perform a variety of functions. The large intestine is about 2 m long and consists of the colon, rectum and the anus. It is said that cancer can occur in the body where mucous membrane exits. However, the sigmoid colon and the rectum are most vulnerable to cancer.

In Korea, the incidence of colon cancer has been dramatically increasing. Moreover, it is the fourth leading cause of cancer-related death among men in Korea, followed by stomach cancer, lung cancer and liver cancer. It is also shown that similar rates of cancer mortality are found in women and the frequency of colon cancer is higher in men than in women. Most cases occur in patients in their 50s, followed by those in their 60s. Furthermore, the age of the greatest incidence of colon cancer in Korea is likely to be 10 years lower than that in the Western world such as the U.S. and Europe. The incidence frequency of colon cancer accounts for 5%-10% in people in their 30s. In addition, colon cancer is likely to occur in the young generation and it is also found mostly in people who have a family history of colon cancer. In fact, the incidence of colon cancer is caused not by heredity but mostly environmental factors. More specifically, the westernization of the diet and particularly excess intake of animal oil and proteins play a greater role in causing colon cancer. Meanwhile, only 5% of colon cancer cases are attributed to hereditary predisposition. In consequence, people with a high risk of developing colon cancer are those who 1) have been affected by colon polyp, 2) have a family history of colon cancer, 3) suffer from ulcerative colitis for a long period of time, or 4) are attacked by incurable anal fistula.

Typically, colon cancer can be classified by the Dukes staging system or the UICC staging system. The systems for staging colon cancer are not determined not by the size of tumor, but largely by the extent of local invasion, and the presence of distant metastasis.

Standards of the Dukes classification and the UICC classification are given in Tables 1 and 2, respectively.

TABLE 1
Description of the Dukes Classification

| | Post-operation5-Year | |
|---|---|---|
| Stages | Survival Rate | Pathological Conditions |
| Dukes A | 90% | Tumour confined to the intestinal |
| | | wall |
| Dukes B | 60 80% | Tumour invading through the |
| | | intestinal wall, but without lymph |
| | | node involvement |
| Dukes C | 20-50% | With lymph node(s) involvement |
| Dukes D | Less than 20% | With distant metastasis to the |
| | | peritoneum, the liver, the lungs, etc. |

TABLE 2
Characteristics of UICC Stage Classification

| Stages | Pathological Conditions |
|---|---|
| 0 | Limited to mucosa |
| 1 | Extending into muscularis propria but not penetrating through it |
| 2 | Penetrating through muscularis propria, but not to adjacent |
| | organs |
| 3 | Penetrating into adjacent organs. Nodes involved |
| 4 | Distant metastatic spread into, e.g., the peritoneum, the liver, the |
| | lungs, etc. |

Considering that there is a slight difference between these two classifications, it is currently recognized that Dukes A corresponds to UICC stage I, Dukes B to UICC stage II, Dukes C to UICC stage III, and Dukes D to UICC stage IV. Particularly, the Dukes staging system is widely used internationally.

When detected at the early stage, colon cancer can be completely cured by endoscopic resection or surgical operation. Further, although metastasized to the liver or the lungs (distant metastasis), colon cancer may still be completely cured through surgical therapy in a period in which a surgery could be administered.

In other words, surgical therapy is the most effective therapy among the currently available therapies. However, if detected too late, cancer spreads to the organs such as the lungs, the liver, the lymph nodes and the peritoneum in which surgical therapy is difficult to apply. For that reason, contrary to the above case, surgical therapy is no use to apply in this case. Consequently, early detection and treatment are indispensable for treating colon cancer effectively.

Considering that there is a possibility that colon cancer may recur after surgical therapy, the patients should have a regular checkup for the recurrence of colon cancer at intervals of 3 to 4 months after surgical operation. Cancer recurrence is likely to occur in the liver, the lungs and the peritoneum rather than in the other organs. The recurrence is also locally observed in the excised site. The recurrence period of colon cancer is shorter than that of other cancers. The site in which the recurrence occurs is completely cured by resecting. Since more than 80% of the recurrent tumors are diagnosed within 3 years after surgical treatment, no recurrence within five years is defined as a criterion for complete cure.

If detected at an early stage, nearly 100% of colon cancer can be completely cured. In the meantime, it is very difficult to detect colon cancer in asymptomatic patients since the patients with colon cancer have no subjective symptoms in the early stage. Accordingly, a periodic checkup should be required to detect colon cancer. An occult blood test is representative of colon cancer screening in detecting colon cancer.

However, the subject cannot be determined to have colon cancer as he or she shows a positive response in this test, Likewise, the indication of all negative responses does not guarantee the absence of colon cancer.

In this regard, it is unreasonable to apply the occult blood test as an accurate diagnostic method in detecting colon cancer. The screening methods of colon cancer currently producing useful diagnostic results are summarized in Table 3, below.

TABLE 3
Colon Cancer Examination

| Examinations | Methods and Properties |
|---|---|
| Colonography | After a thorough cleaning out of the bowels, air, together |
| | with barium, is injected from the anus into the colon, |
| | followed by taking a series of X-ray images which is read |
| | by a radiologist. |
| Colonoscopy | Short colonoscopy for examining S-colon and long |
| | colonoscopy for examining the entire colon. Able to |
| | examine and remove polyps simultaneously. |
| Tumor marker | A method for diagnosing concealed cancer through |
| | blood test. Tumor markers that guarantee the diagnosis |
| | of cancer at an early stage have not yet been found. |
| | CEA is representative of tumor markers, but is positively |
| | detected only from about half of colon cancer patients. |
| | Used as a marker to indicate the progression of colon |
| | cancer and the therapeutic effect of a therapy. |
| Radiologic | Used to examine the progression of primary lesions and |
| Diagnosis | the distal metastasis of the cancer to the liver |

A tumor marker characteristic of a specific cancer makes it possible to detect the cancer in an early stage through blood inspection. However, no tumor markers specific for colon cancer have been discovered yet. Although used for colon cancer, the marker CEA is positive only for about half of the patients as seen in Table 3. Thus, this marker is mainly employed to indicate the progression of colon cancer and the therapeutic effect of a therapy, but the marker is not reliable as a diagnostic marker for the early detection of colon cancer.

AZGP1 (alpha-2-glycoprotein 1, zinc-binding) is a secretary protein which consists of 295 amino acids and has the molecular weight of 33872 Da.

CXCL6 is a secretary protein which consists of 114 amino acids and has the molecular weight of 11,897 Da. It has a chemotactic function against neutrophils and granulocytes.

EGFL6 (EGF-like-domain, multiple 6) consists of 553 amino acids and has the molecular weight of 61317 Da, which is largely detected in fetal tissues. The previous study on the above gene is exemplified by U.S. Pat. No. 6,808,890.

AGT is angiotensinogen (serpin peptidase inhibitor, clade A, member 8) which consists of 485 amino acids and has the molecular weight of 53154 Da. This is also a secretary protein existing as a complex having PRG2 proform comprising disulfide-linked 2:2 heterotetramer, pro-PRG2 and C3 protein during pregnancy.

This protein was detected in pancreatic ductal cancer tissues (Ohta T, Amaya K, Yi S, Kitagawa H, Kayahara M, Ninomiya I, Fushida S, Fujimura T, Nishimura G, Shimizu K, Miwa K. Angiotensin converting enzyme-independent, local angiotensin II—generation in human pancreatic ductal cancer tissues. Int J Oncol. 2003 September; 23(3):593-8) and human male germ cell tumors (Murty V V, Li R G, Mathew S, Reuter V E, Bronson D L, Bosl G J, Chaganti R S. Replication error-type genetic instability at 1q42-43 in human male germ cell tumors. Cancer Res. 1994 Aug. 1; 54(15):3983-5) in relation to cancer.

CXCL 3 (C-X-C chemokine ligand 3) is a secretary protein which consists of 107 amino acids and has the molecular weight of 11342 Da. CXCL 3 exists in extracellular space. It has chemotactic activity against neutrophils and plays an important role in case of inflammation response.

Leading to the present invention, intensive and thorough research through the examination of various genes expected to be involved in colon cancer for expression levels in cancer tissues, such as colon cancer, stomach cancer, breast cancer, prostate cancer, liver cancer, etc. as well as in normal tissues using DNA chips, resulted in the finding that of the genes specifically expressed only in colon cancer tissues, highly putative colon cancer markers were confined to AZGP1, EGFL6, CTHRC1, CXCL3, CXCL-6, AGT, FCGR3A, Col5A2, and S100P, and could be used alone or in combination as diagnostic markers for accurately detecting colon cancer in an early stage.

### DISCLOSURE OF INVENTION

### Technical Problem

It is an object of the present invention to provide a diagnosis marker for colon cancer, which can induce a quantitatively analyzable reaction with at least one protein or gene selected from among proteins or genes of AZGP1, EGFL6, CTHRC1, CXCL3, CXCL-6, AGT, FCGR3A, Col5A2, and S100P.

It is another object of the present invention to provide diagnostic composition of colon cancer, comprising a marker for measuring an mRNA or protein expression level of at least one selected from among AZGP1, EGFL6, CTHRC1, CXCL3, CXCL-6, AGT, FCGR3A, Col5A2, and S100P.

It is a further object of the present invention to provide a diagnostic kit for colon cancer, comprising diagnostic composition of colon cancer. It is still a further object of the present invention to provide a method of yielding information necessary for the diagnosis of colon cancer, using diagnostic composition or kit for the colon cancer.

The present invention also provides a use of the said marker for the production of a composition for the diagnosis of colon cancer.

The present invention also provides a use of the said marker for the production of a kit for the diagnosis of colon cancer

### Technical Solution

In accordance with an aspect thereof, the present invention provides a diagnostic composition of colon cancer, comprising at least one marker for measuring an mRNA expression level of at least one selected from among genes having base sequences of SEQ ID NOS. 1 to 9.

In accordance with another aspect thereof, the present invention provides a diagnostic composition of colon cancer, comprising at least one marker for measuring an expression level of a protein encoded by one gene selected from among genes having base sequences of SEQ ID NOS. 1 to 9.

In another preferred embodiment of the present invention, the present invention provides a use of a marker capable of measuring mRNA expression level of a specific gene selected from the gene group consisting of those genes having the nucleotide sequences represented by SEQ. ID. NO: 1 NO: 9 or a combined marker comprising at least two markers mentioned above for the production of a diagnostic composition of colon cancer.

In another preferred embodiment of the present invention, the present invention provides a use of a marker capable of measuring the expression of a protein encoded by a gene selected from the gene group consisting of those genes having the nucleotide sequences represented by SEQ. ID. NO: 1 NO: 9 or a combined marker comprising at least two markers mentioned above for the production of a composition diagnosis of colon cancer.

The genes serving as diagnosis markers useful in the present invention are AZGP1 (alpha-2-glycoprotein 1) of SEQ ID NO. 1, CXCL3 (C-X-C chemokine ligand 3) of SEQ ID NO. 2, CXCL6 [chemokine (C-X-C motif) ligand 6, granulocyte chemotactic protein 2] of SEQ ID NO. 3, AGT[angiotensinogen(serpin peptidase inhibitor, clade A, member 8)] of SEQ ID NO. 4, FCGR3A of SEQ ID NO. 5, Col5A2 (collagen, type V, alpha 2) of SEQ ID NO. 6, S100P (S100 calcium binding protein P) of SEQ ID NO. 7, EGFL6 (EGF-like-domain, multiple 6) of SEQ ID NO. 8, and CTHRC1 (collagen triple helix repeat containing 1) of SEQ ID NO. 9.

The colon cancer diagnostic composition according to the present invention comprises a marker for measuring the mRNA or protein expression level of at least one selected from among the genes of SEQ ID NOS. 1 to 9. Preferably, the composition comprises two or more markers in combination. In this regard, the markers in combination may be composed of markers capable of measuring an mRNA expression level of one of the genes and a protein expression level of the same gene. Alternatively, the markers in combination are composed of markers capable of measuring mRNA expression levels or protein expression levels of two or more of the genes. When comprising the markers in combination, the composition diagnosis of colon cancer in accordance with the present invention can quantitatively analyze both the mRNA expression levels of the genes and the expression levels of the proteins encoded by the gene at the same time, thereby diagnosing colon caner at an early stage with a high level of reliability.

In an example of the present invention, the expression levels of the genes were found to be two to nine times higher in the biological samples taken from patients with colon cancer than in those taken from normal control.

It should be understood that base sequences showing sequence homology with those of the genes of SEQ ID NOS. 1 to 9 falls within the scope of the present invention. Likewise, the polypeptide sequences showing sequence homology with those encoded by the gene of SEQ ID NOS. 1 to 9 can be used in the present invention.

Sequence homology is used to describe the sequence relationships between two or more nucleic acids, polynucleotides, proteins or polypeptides and is understood in the context of the terms including (a) “reference sequence”, (b) “comparison window”, (c) “sequence identity”, (d) “percentage of sequence identity” and (e) “substantial identity” or “homologous”.

(a) A “reference sequence” is a defined sequence used as a basis for sequence comparison. A reference sequence may be a subset or the entirety of a specified sequence, for example, as a segment of a full-length cDNA or gene sequence, or the complete cDNA or gene sequence.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions or deletions (i.e., gaps) compared to the reference sequence (which does not comprise additions, substitutions or deletions) for optimal alignment of the two sequences. Generally, the comparison window is at least 20 contiguous nucleotides in length, and optionally can be 30, 40, 50, 100, or longer. It is obvious to those skilled in the art that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; (Higgins and Sharp, Gene, 73: 237-244, 1988). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences (See, Current Protocols in Molecular Biology, Chapter 19, Ausubel, et al., Eds., Greene Publishing and Wiley-Interscience, New York (1995). New versions of these or new programs will be obviously available and can be used along with the present invention.

(c) “sequence identity” or “identity” in the context of two nucleic acid or polypeptide sequences includes reference to the residues in the two sequences which are the same when aligned for maximum correspondence over a specified comparison window and which can be mutated typically by addition, deletion or substitution. When percentage of sequence identity is used in reference to proteins, it is recognized that residue positions which are not identical often differ by conservative amino acid substitutions, where amino acid residues are substituted for other amino acid residues with similar chemical properties (e. g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” Means for making this adjustment are well-known to those skilled in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e. g., according to the algorithm of Meyers and Miller, Computer Applic. Biol. Sci., 4: 11-17 (1988) e. g., as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window may comprise additions, substitutions or deletions (gaps) as compared to the reference sequence (which does not comprise additions, substitutions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) i) The term “substantial identity” or “homologous” means that a polynucleotide comprises a sequence that has at least 60% sequence identity, preferably at least 70%, more preferably at least 80%, far more preferably at least 90%, and most preferably 95%, 96%, 97%, 98%, 99% or 100%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like.

Substantial identity of amino acid sequences for these purposes normally means sequence identity of at least 60%, more preferably at least 70%, 80%, 90%, and most preferably at least 95%, 96%, 97%, 98%, 99% or 100%. Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. For example, this may occur when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

(e) ii) The terms “substantial identity” or “homologous” in the context of a peptide indicates that a peptide comprises a sequence with at least 60% sequence identity to a reference sequence, preferably 70%, more preferably 80%, far more preferably 85%, most preferably at least 90% or 95%, 96%, 97%, 98%, 99% or 100% sequence identity to the reference sequence over a specified comparison window.

The term “diagnosis”, as used herein, means the process of identifying a medical condition or disease by its signs and symptoms. For the purpose of the present invention, “diagnosis” is used to mean determining the incidence of colon by examining whether the diagnostic marker of the present invention is expressed.

As used herein, the term “colon cancer” is intended to refer to cancerous growths on the innermost surface mucous membrane, including colon carcinoma, rectal cancer, and anal cancer.

The terms “marker for diagnosis”, “diagnostic marker” or “diagnosis marker”, as used herein, is intended to indicate a substance capable of diagnosing colon cancer by distinguishing colon cancer cells from normal cells, and includes organic biological molecules, quantities of which are increased or decreased in colon cancer cells relative to normal cells, such as polypeptides or nucleic acids (e. g., mRNA, etc.), lipids, glycolipids, glycoproteins and sugars (monosaccharides, disaccharides, oligosaccharides, etc.). Also, primers and antibodies fall within the scope of the markers according to the present invention as long as they can be used to quantitatively measure the change of these biomolecules in expression level in vivo. With respect to the objects of the present invention, examples of the colon cancer diagnostic markers include AZGP1, EGFL6, CTHRC1, CXCL3, CXCL-6, AGT, FCGR3A, Col5A2, and S100P of respective SEQ ID NOS. 1 to 9, which are genes whose expression is increased in colon cancer cells, related nucleic acids (e.g., mRNAs), organic biomolecules such as lipids, glycolipids, glycoproteins, sugars (monosaccharides, disaccharides, oligosaccharides), primer sets or DNA chips capable of identifying the expression patterns of the mRNAs, and antibodies capable of identifying the expression patterns of the proteins.

The terms “Marker for diagnosis”, “diagnostic marker” or “diagnosis marker”, as used herein, is intended to indicate a substance capable of diagnosing colon cancer by distinguishing colon cancer cells from normal cells, and includes organic biological molecules, quantities of which are increased or decreased in colon cancer cells relative to normal cells, such as polypeptides or nucleic acids (e. g., mRNA, etc.), lipids, glycolipids, glycoproteins and sugars (monosaccharides, disaccharides, oligosaccharides, etc.). Also, primers and antibodies fall within the scope of the markers according to the present invention as long as they can be used to quantitatively measure the change of these biomolecules in expression level in vivo. With respect to the objects of the present invention, examples of the colon cancer diagnostic markers include AZGP1, EGFL6, CTHRC1, CXCL3, CXCL-6, AGT, FCGR3A, Col5A2, and S100P of respective SEQ ID NOS. 1 to 9, which are genes whose expression is increased in colon cancer cells, related nucleic acids (e.g., mRNAs), organic biomolecules such as lipids, glycolipids, glycoproteins, sugars (monosaccharides, disaccharides, oligosaccharides), primer sets or DNA chips capable of identifying the expression patterns of the mRNAs, and antibodies capable of identifying the expression patterns of the proteins.

The selection and application of significant diagnostic markers determine the reliability of diagnosis results. A significant diagnostic marker means a marker that has high validity, giving accurate diagnostic results, and high reliability, supplying constant results upon repeated measurement. The colon cancer diagnostic markers of the present invention, which are genes whose expression always increases by direct or indirect factors when colon cancer occurs, display the same results upon repeated tests, and have high reliability due to a great difference in expression levels compared to a control, thus having a very low possibility of giving false results. Therefore, based on the results, the diagnosis, obtained by measuring the expression levels of the significant diagnostic markers of the present invention, is valid and reliable.

At this time, the genes which are expressed on almost the same level between normal colonic epithelial cells and colon cancer cells were excluded. The genes which were expressed at two to nine or more times higher levels specifically in colon cancer cells compared to cells of normal tissues were selected as diagnostic markers of colon cancer.

As long as it is applied to the quantification of mRNA levels of at least one of the genes, any primer set may be used as a diagnostic marker. Preferable is a primer set binding specifically to one of SEQ ID NOS. 1 to 9. In the present invention, the primer set is selected from among base sequence sets of SEQ ID NOS. 10 to 27.

As used herein, the term “primer” refers to a short nucleic acid strand having a free 3′ hydroxyl group, which forms a base pair with a complementary template so as to serve as a starting point for the production of a new template strand. DNA synthesis or replication requires a suitable buffer, proper temperatures, polymerizing enzyme (DNA polymerase, or reverse transcriptase), and four kinds of nucleotide triphosphates, in addition to primers. The primers useful in the present invention are sense and antisense nucleic acids ranging in length from 7 to 50 nucleotides. As long as its basic property of serving as a starting point is not altered, the primers may incorporate an additional characteristic thereinto.

The primers useful in the present invention may be chemically synthesized using a phosphoamidite solid support method or other well-known techniques. Its nucleotide sequences may be modified using various means known in the art. Illustrative, non-limiting examples of the modification include methylation, capping, substitution of natural nucleotides with one or more homologues, and alternation between nucleotides, such as uncharged linkers (e.g., methyl phosphonate, phosphotriester, phosphoroamidate, carbamate, etc.) or charged linkers (e.g., phosphorothioate, phosphorodithioate, etc.). Nucleic acids may contain one or more additionally covalent-bonded residues, which are exemplified by proteins (e. g., nucleases, toxins, antibodies, signal peptides, poly-L-lysine, etc.), intercalating agents (e. g., acridine, psoralene, etc.), chelating agents (e. g., metals, radioactive metals, iron, oxidative metals, etc.), and alkylating agents. The nucleic acid sequences of the present invention may also be altered using a label capable of directly or indirectly supplying a detectable signal. Examples of the label include radioisotopes, fluorescent molecules and biotin.

In accordance with an embodiment of the present invention, the composition for detecting a diagnostic marker of colon cancer includes a pair of primers specific to one or more genes selected from among AZGP1, CXCL3, CXCL6, AGT, FCGR3A, Col5A2, S100P, EGFL6, and CTHRC1 (Table 4).

TABLE 4
[Table 4]
[Table ]

| AZGP1 | | Co15A2 | |
|---|---|---|---|
| SEQ ID NO. | forward ctctgcggaaat- | SEQ ID NO. | forward gacctcgtggt- |
| 10 | acctgaaa | 20 | gacaaaggt |
| SEQ ID NO. | Reverse tgaagaa- | SEQ ID NO. | Reverse agccgcct- |
| 11 | catctccccgtaa | 21 | gatcttcagtaa |
| CXCL3 | | S100P | |
| SEQ ID NO. | forward ggtgctccccttgttcag | SEQ ID NO. | forward agacagc- |
| 12 | | 22 | catgggcatgat |
| SEQ ID NO. | Reverse agggaattcacctcaaga | SEQ ID NO. | Reverset catttgagtcct- |
| 13 | | 23 | gccttctc |
| CXCL6 | | EGFL6 | |
| SEQ ID NO. | forward agatccctggacccagta | SEQ ID NO. | forward gcatgaaaaa- |
| 14 | | 24 | gaaggcaaaa |
| SEQ ID NO. | Reverse ttgccaaagggttcaata | SEQ ID NO. | Reverse tgtcattcttcagggctttc |
| 15 | | 25 | |
| AGT | | CTHRC1 | |
| SEQ ID NO. | forward gctgcaaaacttgacacc | SEQ ID NO. | forward tcatcg- |
| 16 | | 26 | cacttcttctgtgga |
| SEQ ID NO. | Reverse attgcctgtagcctgtca | SEQ ID NO. | Reverse gccaaccca- |
| 17 | | 27 | gatagcaacatc |
| FCGR3A | | β-actin (Control) | |
| SEQ ID NO. | forward gcttgttgggag- | A | forward gatcattgctc- |
| 18 | taaaaatg | | ctcctgagc |
| SEQ ID NO. | Reverse tccagtcttgttgagctt | B | Reverse actcct- |
| 19 | gcttgctgatccac | | |

In the composition for the diagnosis of colon cancer according to the present invention, any can be used as a marker for measuring expression levels of the proteins as long as it detects a change of proteins in expression level in colon cancer cells. Preferably, the marker is an antibody specific for one of the proteins encoded by the gene of SEQ ID NOS. 1 to 9 (AZGP1, CXCL3, CXCL6, AGT, FCGR3A, Col5A2, S100P, EGFL6, and CTHRC1).

The term “antibody” as used herein, refers to a specific protein molecule that indicates an antigenic region. With respect to the objects of the present invention, an antibody binds specifically to a marker protein, and includes all of polyclonal antibodies, monoclonal antibodies and recombinant antibodies.

Since the colon cancer marker protein is identified as described above, it may be used to produce antibodies using techniques widely known in the art.

Polyclonal antibodies may be produced by a method widely known in the art, which includes injecting the colon cancer marker protein antigen into an animal and collecting blood samples from the animal to obtain serum containing antibodies. Such polyclonal antibodies may be prepared from a certain animal host, such as goats, rabbits, sheep, monkeys, horses, pigs, cows and dogs. The antibodies produced can be isolated and purified using gel electrophoresis, dialysis, salting out, ion exchange chromatography, affinity chromatography, and other techniques.

Monoclonal antibodies may be prepared by a method widely known in the art, such as a hybridoma method (Kohler and Milstein (1976) European Journal of Immunology 6:511-519), or a phage antibody library technique (Clackson et al., Nature, 352:624-628, 1991; Marks et al, J. Mol. Biol., 222:58, 1-597, 1991). The antibody produced above can be isolated and purified by gel electrophoresis, dialysis, salt precipitation, ion exchange chromatography, affinity chromatography, etc.

In addition, the antibodies of the present invention include complete forms having two full-length light chains and two full-length heavy chains, as well as functional fragments of antibody molecules. The functional fragments of antibody molecules refer to fragments retaining at least an antigen-binding function, and include Fab, F(ab′), F(ab′)2, Fv and the like.

In the composition for the diagnosis of colon cancer, the antibody is preferably a microparticle-conjugated antibody. The micro particle may be preferably colored latex or colloidal gold particle.

In the composition for the diagnosis of colon cancer, any antibody may be used as long as it can be applied to the quantitative analysis of the expression level of the proteins encoded by the genes of SEQ ID NOS. 1 to 9. Preferable is an antibody used in an immunochromatographic strip kit, a Luminex assay kit, a protein microarray kit, an ELISA kit or an immunodot kit.

Preferably, the immunochromatographic strip useful in the composition for the diagnosis of colon cancer comprises (a) a sample pad onto which a sample is absorbed; (b) a conjugate pad in which an antibody binds to proteins encoded by one or more genes selected from among base sequences of SEQ ID NOS. 1 to 9; (c) a test membrane with a test line and a control line, comprising a monoclonal antibody to the proteins encoded by one or more selected from among the genes of SEQ ID NOS. 1 to 9; (d) an absorbent pad into which remaining samples are absorbed; and (e) a support.

The Luminex assay kit, the microarray kit, or the ELISA kit which may be useful in the composition for the diagnosis of colon cancer preferably comprises a secondary antibody the poly- or monoclonal antibody, whether conjugated with a label, to a protein encoded by the gene selected from among the genes of SEQ ID NOS. 1 to 9.

In accordance with another aspect thereof, the present invention provides a kit for diagnosing colon cancer, comprising the colon cancer diagnostic composition containing one or more markers capable of measuring the expression level of mRNA or protein of the gene selected from among genes of SEQ ID NOS. 1 to 9.

In another preferred embodiment of the present invention, the present invention provides a use of a marker capable of measuring mRNA expression level of a specific gene selected from the gene group consisting of those genes having the nucleotide sequences represented by SEQ. ID. NO: 1 NO: 9 or a combined marker comprising at least two markers mentioned above for the production of a kit for the diagnosis of colon cancer.

In another preferred embodiment of the present invention, the present invention provides a use of a marker capable of measuring the expression of a protein encoded by a gene selected from the gene group consisting of those genes having the nucleotide sequences represented by SEQ. ID. NO: 1 NO: 9 or a combined marker comprising at least two markers mentioned above for the production of a kit for the diagnosis of colon cancer.

The term “measurement of mRNA expression levels” or corresponding phrases, as used herein, are intended to refer to a process of assessing the presence and expression levels of mRNA of colon cancer marker genes in biological samples for diagnosing colon cancer, in which the amount of mRNA is measured. Analysis methods for measuring mRNA levels include, but are not limited to, RT-PCR, competitive RT-PCR, real-time RT-PCR, RNase protection assay (RPA), Northern blotting and DNA chip assay.

The term “measurement of protein expression levels” or corresponding phrases, as used herein, are intended to refer to a process of assessing the presence and expression levels of proteins expressed from colon cancer marker genes in biological samples for diagnosing colon cancer, in which the amount of protein products of the marker genes is measured using antibodies specifically binding to the proteins. Analysis methods for measuring protein levels include, but are not limited to, Western blotting, enzyme linked immunosorbent assay (ELISA), radioimmunoassay (RIA), radioimmunodiffusion, Ouchterlony immunodiffusion, rocket immunoelectrophoresis, immunohistostaining, immunoprecipitation assay, complement fixation assay, FACS, and protein chip assay.

In a preferable embodiment, the diagnostic kit of the present invention is characterized by including essential elements required for performing RT-PCR. An RT-PCR kit includes a pair of primers specific for each marker gene. The primers are nucleotides having sequences specific to a nucleic acid sequence of each marker gene, and are about 7 by to 50 bp in length, more preferably about 10 by to 30 bp in length. Also, the RT-PCR kit may include primers specific to a nucleic acid sequence of a control gene. The RT-PCR may further include test tubes or other suitable containers, reaction buffers (varying in pH and magnesium concentrations), deoxynucleotides (dNTPs), enzymes such as Taq-polymerase and reverse transcriptase, DNAse, RNAse inhibitor, DEPC-treated water, and sterile water.

As long as it is applied to diagnose colon cancer, any type kit can be used in the present invention. Preferable is a reverse transcription-polymerase chain reaction kit, an immunodot kit, an ELISA kit, an immunochromatography kit, a Luminex assay kit, or a protein microarray kit thanks to their ability to rapidly and accurately measure mRNA or protein expression levels of biological samples. Preferably, a diagnostic kit for the colon cancer may further comprise one or more components, solutions or devices suitable for the analysis of colon cancer.

The luminex kit useful as a diagnostic kit of the present invention may comprise poly- and monoclonal antibodies to the proteins encoded by the genes of SEQ ID NOS. 1 to 9, and a secondary antibody to the poly- or monoclonal antibodies. The luminex assay according to the present invention is high-throughput quantification method which can analyze as many as 100 analytes at the same time even if the patient samples are present in a small amount (10˜20 μl) and are not pretreated. The luminex assay is highly sensitive (pg level) and can perform quantitative analysis within a short time (3˜4 hours), so that it is used as an alternative to ELISA or ELISPOT assay. An luminex assay is a multiplexed fluorescent microplate method by which 100 or more biological samples can be analyzed in each well of 96-well plates and employs two laser detectors to progress signal transmission in real time, so that polystyrene beads can be discriminated by 100 or more colors. 100 beads are designed in the following manner. In a 10×10 bead matrix, red fluorescent beads and orange fluorescent beads are divided into 10 or more classes according to intensities on respective sides. Within the matrix, the columns contain beads at different ratios of red and orange colors to form 100 color-coded bead set in total. Also, each bead is coated with an antibody to a target protein and thus can be used for protein quantification through immune responses. In this assay, a sample is analyzed using two laser rays. One laser is used to detect beads to identify the inherent bead number provided while the other laser functions to sense a sample protein reacted with the antibody conjugated to the bead. Therefore, 100 different proteins can be analyzed at the same time in one well. This assay also enjoys the advantage of sensing a sample even if it is present in an amount of as small as 15 μl.

A luminex kit with which a luminex assay can be performed in accordance with the present invention includes an antibody specific to the marker protein. The antibody may be a monoclonal, polyclonal or recombinant antibody, which has high specificity and affinity to each marker protein and rarely has cross-reactivity to other proteins. Also, the Luminex kit may comprise an antibody specific for a control protein. The Luminex kit may further include reagents capable of detecting bound antibodies, for example, a labeled secondary antibody, chromophores, enzymes (e. g., conjugated with an antibody) and their substrates or other substances capable of binding to the antibodies. Also, the antibody may be an antibody conjugated to microparticles which may be selected from among colored latex particles and colloidal gold particles.

In another embodiment of the present invention, the diagnostic kit may be characterized by including essential elements required for performing a DNA chip assay. A DNA chip kit may include a substrate plate onto which genes or fragments thereof, cDNA or oligonucleotides, are attached, and reagents, agents and enzymes for preparing fluorescent probes. Also, the substrate plate may include a control gene or fragments thereof, such as cDNA or oligonucleotides.

Further, preferably, the diagnostic kit is characterized by including essential elements required for performing ELISA. An ELISA kit includes antibodies specific to marker proteins. The antibodies may be monoclonal, polyclonal or recombinant antibodies, which have high specificity and affinity to each marker protein and rarely have cross-reactivity to other proteins. Also, the ELISA kit may include an antibody specific to a control protein. The ELISA kit may further include reagents capable of detecting bound antibodies, for example, a labeled secondary antibody, chromophores, enzymes (e. g., conjugated with an antibody) and their substrates or other substances capable of binding to the antibodies.

The diagnostic kit for colon cancer comprising an immunochromatographic strip for diagnosing colon cancer is characterized by including essential elements required for performing a rapid diagnostic test which gives an analysis result within 5 min. A rapid diagnostic test kit with an immunochromatographic strip includes antibodies specific to marker proteins. The antibodies may be monoclonal, polyclonal or recombinant antibodies, which have high specificity and affinity to each marker protein and rarely have cross-reactivity to other proteins. Also, the rapid test kit may further include other substances necessary for the diagnosis, for example, a membrane on which specific antibodies and secondary antibodies are immobilized, a membrane with antibody-conjugated beads bound thereto, an absorbent pad, and a sample pad.

Also, the colon cancer diagnostic kit of the present invention may be characterized by including essential elements required for performing protein microarray for analyzing combined markers simultaneously. The protein microarray kit useful in the present invention includes antibodies specific to marker proteins bound to a solid support. The antibodies may be monoclonal, polyclonal or recombinant antibodies, which have high specificity and affinity to each marker protein and have little cross-reactivity to other proteins. Also, the protein microarray kit may include an antibody specific to a control protein. The protein microarray kit may further include reagents capable of detecting bound antibodies, for example, a labeled secondary antibody, chromophores, enzymes (e. g., conjugated with an antibody) and their substrates or other substances capable of binding to the antibodies. The protein microarray of the present invention may include poly- and/or monoclonal antibodies to the protein bound to the slide and an enzyme-conjugated secondary antibody to the poly- or monoclonal antibodies.

In another preferred embodiment of the present invention, the present invention provides a method for the diagnosis of colon cancer among patients having high risk of colon cancer, which is composed of the following steps:

1) measuring expression levels of one or more genes selected from the gene group consisting of those genes having the nucleotide sequences represented by SEQ. ID. NO: 1 NO: 9 in biological samples taken from patients; and

2) taking the measured expression levels, particularly increased levels, as colon cancer risk index, selecting patients demonstrating higher expression levels than normal people.

The expression level herein indicates the level of mRNA of one or more genes selected from those genes having the nucleotide sequences represented by SEQ. ID. NO: 1 NO: 9 or the expression level of a protein expressed from one or more genes selected from the group consisting of those genes having the nucleotide sequences represented by SEQ. ID. NO: 1 NO: 9.

In accordance with another aspect thereof, the present invention provides a method for yielding information necessary for the diagnosis of colon cancer, comprising measuring mRNA levels in a biological sample from a patient with suspected colon cancer using one or more primer sets, selected from among base sequences of SEQ ID NOS. 10 to 27, specific to one or more genes selected from among genes of SEQ ID NOS. 1 to 9 (AZGP1, CXCL3, CXCL6, AGT, FCGR3A, Col5A2, S100P, EGFL6, and CTHRC1); and comparing mRNA levels of the sample from the patient with those of a normal control sample to determine an increase in mRNA levels.

The isolation of mRNA from a biological sample may be achieved using a known process, and mRNA levels may be measured by a variety of methods.

Analysis methods for measuring mRNA levels include RT-PCR, competitive RT-PCR, real-time RT-PCR, RNase protection assay (RPA), Northern blotting and DNA chip assay, but are not limited thereto.

With the detection methods, a patient with suspected colon cancer is compared with a normal control for mRNA expression levels of a colon cancer marker gene, and the patient's suspected colon cancer is diagnosed by determining whether expression levels of mRNA from the colon cancer marker gene have significantly increased.

mRNA expression levels are preferably measured by RT-PCR or DNA chip using primers specific to a gene serving as a colon cancer marker.

After RT-PCT, the products are electrophoresed, and patterns and thicknesses of bands are analyzed to determine the expression and levels of mRNA from a gene used as a diagnostic marker of colon cancer while comparing the mRNA expression and levels with those of a control, thereby simply diagnosing the incidence of colon cancer. Alternatively, mRNA expression levels may be measured using a DNA chip in which the colon cancer marker genes or nucleic acid fragments thereof are anchored at high density to a glass-like base plate. A cDNA probe labeled with a fluorescent substance at its end or internal region is prepared using mRNA isolated from a sample, and is hybridized with the DNA chip. The DNA chip is then read to determine the presence or expression levels of the gene, thereby diagnosing the incidence of colon cancer.

In accordance with another aspect thereof, the present invention provides a method of diagnosing colon cancer, comprising measuring protein levels by contacting an antibody specific to one or more genes selected from among the genes of SEQ ID NOS. 1 to 9(AZGP1, CXCL3, CXCL6, AGT, FCGR3A, Col5A2, S100P, EGFL6, and CTHRC1) with a biological sample from a patient with suspected colon cancer to form antigen-antibody complexes; and comparing protein levels of the sample from the patient with those of a normal control sample to determine an increase in protein level.

The isolation of proteins from a biological sample may be achieved using a known process, and protein levels may be measured by a variety of methods.

Analysis methods for measuring mRNA levels include RT-PCR, competitive RT-PCR, real-time RT-PCR, RNase protection assay (RPA), Northern blotting and DNA chip assay, but are not limited thereto.

The term “biological sample”, as used herein particularly for the measurement of mRNA or protein levels, includes samples displaying a difference in expression levels of a colon cancer marker gene, such as tissues, cells, whole blood, serum, plasma, saliva, sputum, cerebrospinal fluid and urine, but is not limited thereto.

Analysis methods for measuring protein levels in accordance with the present invention include, but are not limited to, an immunochromatography assay, an immunodot assay, a Luminex assay, an ELISA assay, a protein microarray assay, an immunostaining assay, a Western blotting assay, a radioimmunoassay (RIA), a radioimmunodiffusion assay, an ouchterlony immunodiffusion assay, a rocket immunoelectrophoresis assay, an immunohistostaining assay, an immunoprecipitation assay, a complement fixation assay, FACS, and a protein chip assay.

The measurement of protein levels by immunodot assay may be carried out by (a) dotting a biological sample on a membrane; (b) reacting the sample with antibodies specific for the proteins encoded by one or more genes selected from among the genes of SEQ ID NOS. 1 to 9; and (c) adding a labeled secondary antibody to the membrane and developing a color. The ELISA assay is preferably a sandwich ELISA assay which can be implemented by (a) immobilizing Antibody 1 to the proteins of one or more genes selected from among the genes of SEQ ID NOS. 1 to 9; (b) reacting the immobilized Antibody 1 with a biological sample from a patient with suspected colon cancer to form an antigen-antibody complex; binding to the complex labeled Antibody 2 specific for the proteins encoded by one or more genes selected from among the genes of SEQ ID NOS. 1 to 9; and detecting the label to determine the protein level. The protein microarray assay preferably comprises (a) immobilizing onto a chip a polyclonal antibody specific for the proteins encoded by one or more genes selected from among the genes of SEQ ID NOS. 1 to 9; (b) reacting the immobilized Antibody 1 with a biological sample from a patient with suspected colon cancer to form an antigen-antibody complex; (c) binding to the complex a labeled monoclonal antibody specific for the proteins encoded by one or more genes selected from among the genes of SEQ ID NOS. 1 to 9; and (d) detecting the label to determine the protein level.

Through the analysis assays, a quantitative comparison can be made between the antigen-antibody complexes in a normal control and a patient with suspected colon cancer. Based on this comparison, a significant increase in the level of the colon cancer marker gene can be determined, thus giving information necessary for the diagnosis of colon cancer.

As used herein, the term “antigen-antibody complex” is intended to refer to binding products of a colon cancer marker protein to an antibody specific thereto. The antigen-antibody complex thus formed may be quantitatively determined by measuring the signal size of a detection label.

Such a detection label may be selected from a group consisting of enzymes, fluorescent substances, ligands, luminescent substances, microparticles, redox molecules and radioactive isotopes, but the present invention is not limited to the examples. Examples of the enzymes available as detection labels include, but are not limited to, β-glucuronidase, β-D-glucosidase, β-D-galactosidase, urase, peroxidase, alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphenolpyruvate decarboxylase, and β-latamase. Examples of the fluorescent substances include, but are not limited to, fluorescin, isothiocyanate, rhodamine, phycoerythrin, phycocyanin, allophycocyanin, o-phthaldehyde, fluorescamin and DAP. As the ligands, bitine derivatives are useful, but are not given as a factor limiting the present invention. Examples of the luminescent substances include acridinium esters, luciferin and luciferase, but are not limited thereto. As for the microparticles, its examples include, but are not limited to, colloidal gold and colored latex. Examples of the redox molecules include, but are not limited to, ferrocene, ruthenium complexes, viologen, quinone, Ti ions, Cs ions, diimide, 1,4-benzoquinone, hydroquinone, K₄W(CN)₈, [Os(bpy)₃]²⁺, [RU(bpy)₃]²⁺ and [MO(CN)₈]⁴. Examples of the radioactive isotopes include, but are not limited to, ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I and ¹⁸⁶Re.

Preferably, the protein expression levels are measured by ELISA. Examples of ELISA include direct ELISA using a labeled antibody recognizing an antigen immobilized on a solid support; indirect ELISA using a labeled antibody recognizing a capture antibody forming complexes with an antigen immobilized on a solid support; direct sandwich ELISA using a labeled antibody recognizing an antigen bound to a antibody immobilized on a solid support; and indirect sandwich ELISA, in which a captured antigen bound to an antibody immobilized on a solid support is detected by first adding an antigen-specific antibody, and then a secondary labeled antibody which binds the antigen-specific antibody. More preferably, the protein expression levels are detected by sandwich ELISA, where a sample reacts with an antibody immobilized on a solid support, and the resulting antigen-antibody complexes are detected by adding a labeled antibody specific for the antigen, followed by enzymatic development, or by first adding an antigen-specific antibody and then a secondary labeled antibody which binds to the antigen-specific antibody, followed by enzymatic development. Information necessary for the diagnosis of colon cancer can be provided by measuring the degree of complex formation of a colon cancer marker protein and an antibody thereto.

Further, the measurement of protein expression levels is preferably achieved using Western blotting using one or more antibodies to the colon cancer makers. Total proteins are isolated from a sample, separated according to size by electrophoresis, transferred onto a nitrocellulose membrane, and reacted with an antibody. The amount of proteins produced by gene expression is determined by measuring the amount of antigen-antibody complexes produced using a labeled antibody, thereby diagnosing the incidence of colon cancer. The detection method comprises assessing expression levels of maker genes in a control and cells in which colon cancer occurs. mRNA or protein levels may be expressed as an absolute (e.g., μg/ml) or relative (e. g., relative intensity of signals) difference in the amount of marker proteins.

Also, the measurement of protein expression levels is preferably performed with an immunochromatography diagnostic kit which is characterized by essential elements required for a rapid test which gives a result within 5 min. A rapid test kit using an immunochromatographic strip comprises an antibody specific for a marker protein. The antibody may be a monoclonal, polyclonal or recombinant antibody, which has high specificity and affinity to each marker protein and rarely have cross-reactivity to other proteins.

In addition, the rapid test kit may further include other reagents capable of detecting bound antibodies, for example, a nitrocellulose membrane onto which specific antibodies and secondary antibodies are immobilized, a membrane with antibody-conjugated beads bound thereto, an absorbent pad, and a sample pad.

In addition, the measurement of protein expression levels can be carried out with an assay kit which is characterized by including essential elements required for Luminex assay which is typically designed to analyze combined markers at the same time. A Luminex kit includes an antibody specific for a maker protein. The antibody may be a monoclonal, polyclonal or recombinant antibody, which has high specificity and affinity to each marker protein and rarely have cross-reactivity to other proteins. Also, the Luminex kit may comprise an antibody specific for a control protein. The Luminex kit may further include reagents capable of detecting bound antibodies, for example, a labeled secondary antibody, chromophores, enzymes (e. g., conjugated with an antibody) and their substrates or other substances capable of binding to the antibodies.

The diagnostic kit useful in measuring protein expression levels in accordance with the present invention is characterized by including essential elements required for performing protein microarray so as to analyze combined markers simultaneously. The microarray kit includes antibodies specific to marker proteins bound to a solid support. The antibodies may be monoclonal, polyclonal or recombinant antibodies, which have high specificity and affinity to each marker protein and have little cross-reactivity to other proteins. Also, the protein microarray kit may include an antibody specific to a control protein. The protein microarray kit may further include reagents capable of detecting bound antibodies, for example, a labeled secondary antibody, chromophores, enzymes (e. g., conjugated with an antibody) and their substrates or other substances capable of binding to the antibodies. By a method of analyzing a sample using a protein microassay, proteins are isolated from the sample and hybridized with the protein chip to form antigen-antibody complexes. The protein chip is then read to determine the presence or expression levels of the proteins, thereby providing information necessary for the diagnosis of colon cancer.

In a preferable embodiment, the protein expression levels may be measured through immunohistostaining using one or more antibodies to the colon cancer marker. Normal colonic epithelial tissues and colon cancer-suspected tissues are taken, immobilized, and embedded in a paraffin block which is then sectioned to slices of micrometers thickness on glass slides, followed by reaction with one of the antibodies. Thereafter, the antibodies which remain unreacted are washed off, and the bound antibodies are labeled with one of the above-mentioned detection labels. Under a microscope, the labeling of the antibodies is read.

### Advantageous Effects

The marker of the present invention for the diagnosis of colon cancer facilitates fast and easy diagnosis of colon cancer by using those genes over-expressed specifically in colon cancer tissues and therefore it can be effectively used for the screening of candidates for colon cancer treatment agents.

### BRIEF DESCRIPTION OF DRAWINGS

FIG. 1 is electrophoresis photographs showing expression levels of AZGP1, AGT, EGFL6, and CXCL3 in normal tissues and colon cancer tissues as identified by reverse transcription PCR.

FIG. 2 is electrophoresis photographs showing expression level of CTHRC1 in normal tissues and colon cancer tissues as identified by reverse transcription PCR.

FIG. 3 is an electrophoresis photograph showing expression levels of AZGP1, AGT, and EGFL6 in 10 colon cancer cell lines as identified by RT-PCR.

FIG. 4 is a view showing expression levels of AGT, EGFL6, and CXCL3 in normal sera and colon cancer sera as identified by Western blotting.

FIG. 5 is microphotographs showing expression level of EGFL6 in normal mucous membrane and colon cancer tissues as identified by immunohistostaining.

FIG. 6 is microphotographs showing expression level of CTHRC1 in normal mucous membrane and colon cancer tissues as identified by immunohistostaining.

FIG. 7 is microphotographs showing expression level of CXCL-3 in normal mucous membrane and colon cancer tissues as identified by immunohistostaining.

FIG. 8 is microphotographs showing expression level of AGT in normal mucous membrane and colon cancer tissues as identified by immunohistostaining.

FIG. 9 is a diagram illustrating the principal of immunological dot assay.

FIG. 10 is a photograph illustrating the comparison of protein expressions between normal serum and colon cancer patient serum, investigated by immunological dot assay.

FIG. 11 is a standard curve for AGT protein, established by an ELISA assay.

FIG. 12 is a schematic diagram showing a structure of an immunochromatographic strip according to the present invention.

### MODE FOR THE INVENTION

A better understanding of the present invention may be obtained through the following examples which are set forth to illustrate, but are not to be construed as limiting the present invention.

### EXAMPLE 1

### Excavation of Genes Overexpressed in Colon Cancer Using DNA Chip

In order to primarily extract genes which are overexpressed specifically in colon cancer cells compared to normal colonic epithelial cells, 2,230 genes were examined for expression level using DNA chips (48K human microarray, commercially available from Illumina).

Total mRNA was isolated from normal colonic epithelial cells and colon cancer cells using an RNeasy Mini Kit (QIAGEN) and quantitatively analyzed on a chip (Experion RNA StdSens, Bio-Rad). For use in hybridization, the total mRNA was biotinylated and amplified using Illumina TotalPrep RNA Amplification Kit (Ambion). cDNA was synthesized with T7 oligo-dT primers and biotinylated by in vitro transcription with biotin-UDP.

The biotin-labeled cDNA thus formed was quantified using NonoDrop. The cDNA prepared from normal colonic epithelial cells and colon cancer cells was hybridized on a chip (Human-6 V2, Illumina). After hybridization, the DNA chip was washed with buffer (Illumina Gene Expression System Wash Buffer, Illumina) to remove non-specific hybridizations and labeled with fluorescent streptavidin-Cy3 conjugate (Amersham).

The fluorescence-labeled DNA chip was scanned using a confocal laser scanner (Illumina) to give fluorescence data of each spot. The fluorescence data were saved as TIFF images. The TIFF images were quantified with BeadStudio version 3 (Illumina) to quantify the fluorescence intensity at each spot. The quantitative results were normalized using the quantile function supplied by the program Avadis Prophetic version 3.3 (Strand Genomics).

As a result, 1,601 genes were analyzed for expression level in normal colonic epithelial cells and colon cancer cells, and the genes with overexpression of mRNA in colon cancer cells were finally selected (Table 5).

TABLE 5

| | KRIBB |
|---|---|
| | Fold change 2ⁿ |
| I AZGP1 | 3.00 |
| EGFL6 | 2.97 |
| S100P | 3.25 |
| CTHRC1 | 2.69 |
| CXCL6 | 2.67 |
| CXCL3 | 0.27 |
| FCGR3A | 0.26 |
| AGT | 2.38 |
| Col5A2 | 1.14 |

### EXAMPLE 2

### mRNA Isolation from Tissues and Cells

For use in reverse transcription PCR, mRNA was isolated from total 40 tissues consisting of normal colonic epithelial cells and colon cancer cell tissues from 20 patients with colon cancer.

First, immediately after the surgical resection of tissues, blood was removed from the tissues in sterile phosphate buffered saline and frozen in liquid nitrogen. Thereafter, total mRNA was isolated in a single-step RNA isolation manner using the guanidinium method. The total mRNA thus obtained was quantified with a spectrophotometer and stored in a −70° C. freezer until use.

10 colon cancer cell lines (DLD-1, HT29. HCT116, colo205, SW480, SW620, SNU C1, SNU C2A, KM 12C, KM 12SM) were obtained from KCLB (the Korean Cell Line Bank, located at 28, Yeonkun-dong, Jongno, Seoul, Korea).

Each cell line was cultured for 5˜6 days in DMEM (Invitrogen) or RPMI1640 (Invitrogen), supplemented with 10% fetal bovine serum (FBS, Hyclon) and 1 mg/ml penicillin/streptomycin (Sigma), after which total RNA was isolated in a single-step RNA isolation manner using the guanidinium method. The RNA thus obtained was quantified with a spectrophotometer and stored at a −70° C. freezer until use.

### EXAMPLE 3

### Comparison of Gene Expression Levels by RT-PCR

The colon cancer-specific, overexpressed genes selected in Example 1 were subjected to RT-PCR.

An overall DNA sequence of each gene was obtained from the NCBI Core Nucleotide database (Core Nucleotide, http://www.ncbi.nlm.nih.gov/). Based on the DNA sequences, primer sequences for the genes were designed using the Primer3 program. PCR was performed with these designed primers to examine expression levels of the genes. Base sequences of the primers are listed in Table 6, below.

TABLE 6
[Table 6]
[Table ]

| AZGP1 | | Co15A2 | |
|---|---|---|---|
| SEQ ID NO. | forward ctctgcggaaat- | SEQ ID NO. | forward gacctcgtggt- |
| 10 | acctgaaa | 20 | gacaaaggt |
| SEQ ID NO. | Reverse tgaagaa- | SEQ ID NO. | Reverse agccgcct- |
| 11 | catctccccgtaa | 21 | gatcttcagtaa |
| CXCL3 | | S100P | |
| SEQ ID NO. | forward ggtgctccccttgttcag | SEQ ID NO. | forward agacagc- |
| 12 | | 22 | catgggcatgat |
| SEQ ID NO. | Reverse | SEQ ID NO. | Reverset catttgagtcct- |
| 13 | agggaattcacctcaaga | 23 | gccttctc |
| CXCL6 | | EGFL6 | |
| SEQ ID NO. | forward agatccctggacccagta | SEQ ID NO. | forward gcatgaaaaa- |
| 14 | | 24 | gaaggcaaaa |
| SEQ ID NO. | Reverse ttgccaaagggttcaata | SEQ ID NO. | Reverse tgtcattcttcagggctttc |
| 15 | | 25 | |
| AGT | | CTHRC1 | |
| SEQ ID NO. | forward gctgcaaaacttgacacc | SEQ ID NO. | forward tcatcg- |
| 16 | | 26 | cacttcttctgtgga |
| SEQ ID NO. | Reverse attgcctgtagcctgtca | SEQ ID NO. | Reverse gccaaccca- |
| 17 | | 27 | gatagcaacatc |
| FCGR3A | | β-actin (Control) | |
| SEQ ID NO. | forward gcttgttgggag- | A | forward gatcattgctc- |
| 18 | taaaaatg | | ctcctgagc |
| SEQ ID NO. | Reverse tccagtcttgttgagctt | B | Reverse actcct- |
| 19 | | | gcttgctgatccac |

Through RT-PCR, the mRNA isolated from the tissues and cell lines of Example 2 were converted into cDNA. In this regard, the cDNA construction was accomplished using a cDNA synthesis kit (AccuScript High Fidelity 1^{st} Stand cDNA Synthesis Kit, STRATAGENE).

From the cDNA, PCR amplification was carried out in the presence of the designed primers (1^{st} cycle: 94° C., 5 min; 2^{nd} to 35^{th} cycles: 94° C., 40 sec, 56° C., 40 sec, 72° C., 30 sec; final extension: 72° C., 7 min).

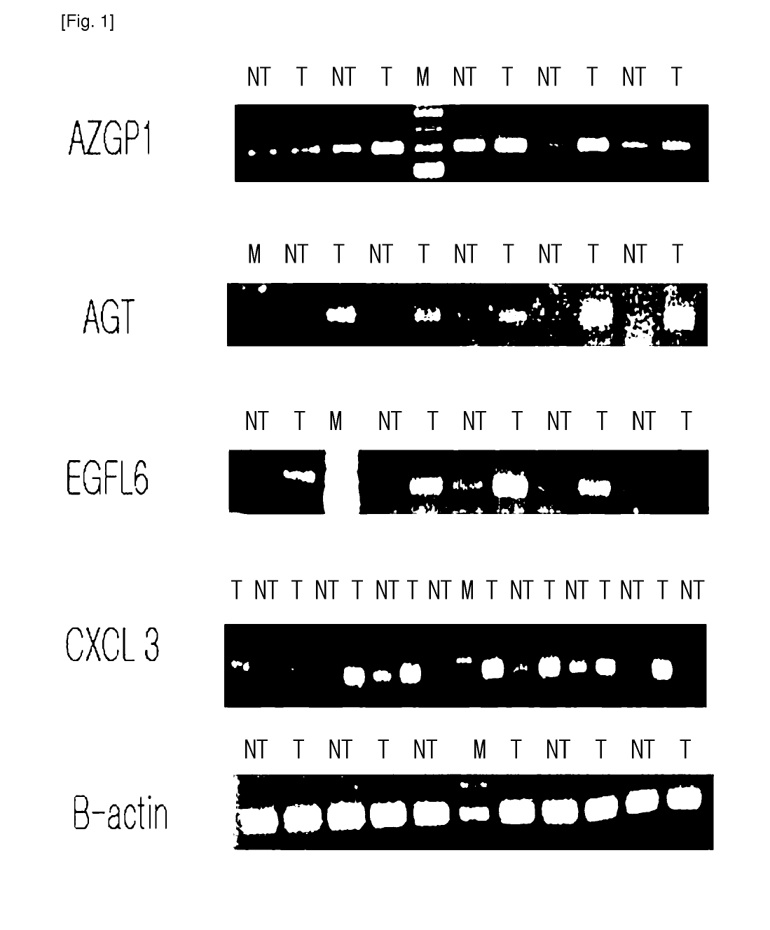

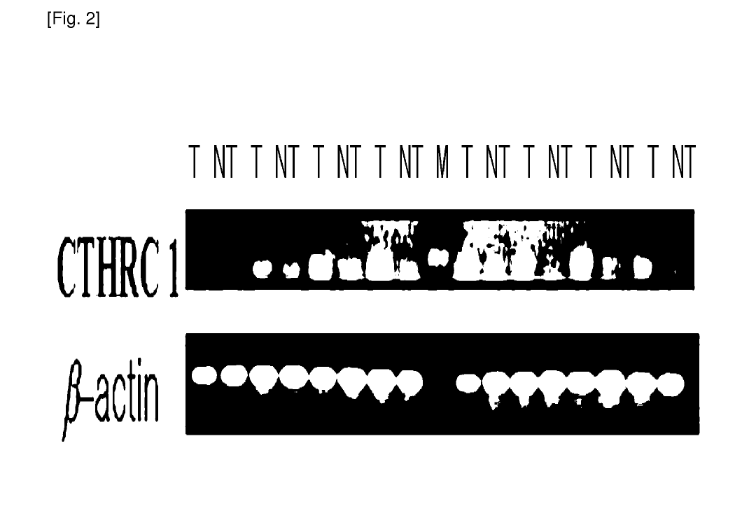

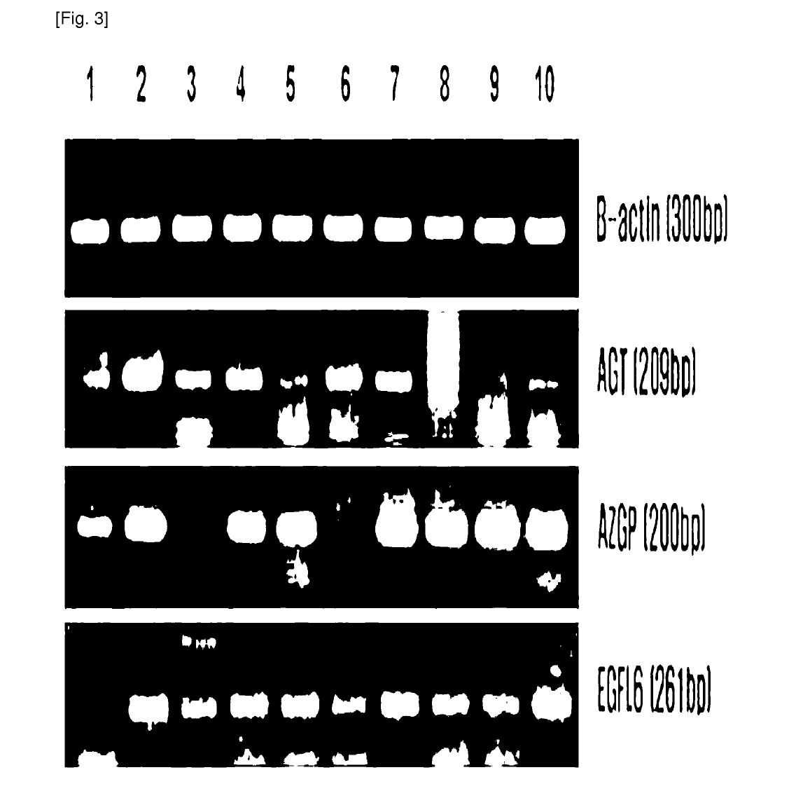

As a result, differences in gene expression level between normal colon cells and colon cancer cells were detected. Coincident with the results of Example 1, the genes of SEQ ID NOS. 1 to 9 was identified to increase their expression levels in the colon cancer cell lines as compared to the normal colon cells (FIGS. 1 to 3).

### EXAMPLE 4

### Comparison of Protein Expression Levels in Sera Using Western Blotting

Protein levels in serum of colon cancer patients and healthy persons were compared using a Western blotting method.

Sera was isolated from the blood of colon cancer patients and healthy persons and diluted with the same volume of a sample buffer (125 mM Tris pH 6.8, 4% SDS, 10% glycerol, 0.006% bromophenol blue, 1.8% BME) before boiling. 12% SDS-PAGE separated serum proteins. The SDS-PAGE gel in which the serum proteins were separated according to sizes was brought into contact with a nitrocellulose membrane. The application of a current to the gel-membrane associate transferred the proteins onto the membrane which was then blocked for 1 hour in a TBST solution (10 mM Tris, 100 mM NaCl, 0.05% Tween 20) containing 3% FBS albumin, followed by reaction with an AGT antibody (R&D, 1:2000) at 4° C. with shaking overnight. Afterwards, excess antibodies were washed off with PBST, and a horse radish peroxydase-conjugated secondary antibody (ABCAM, Rabbit polyclonal to Mouse IgG) was added and incubated at 4° C. for 1 hour with shaking. The nitrocellulose membrane was immersed in a mixture of 1:1 ECL Solution A (containing Luminol and enhancer):Solution B (containing hydrogen peroxide) and incubated for 1 min with shaking. After being dried suitably, the membrane was attached to a film cassette and developed in a dark room. The same procedure was applied to EGFL6 and CXCL-3.

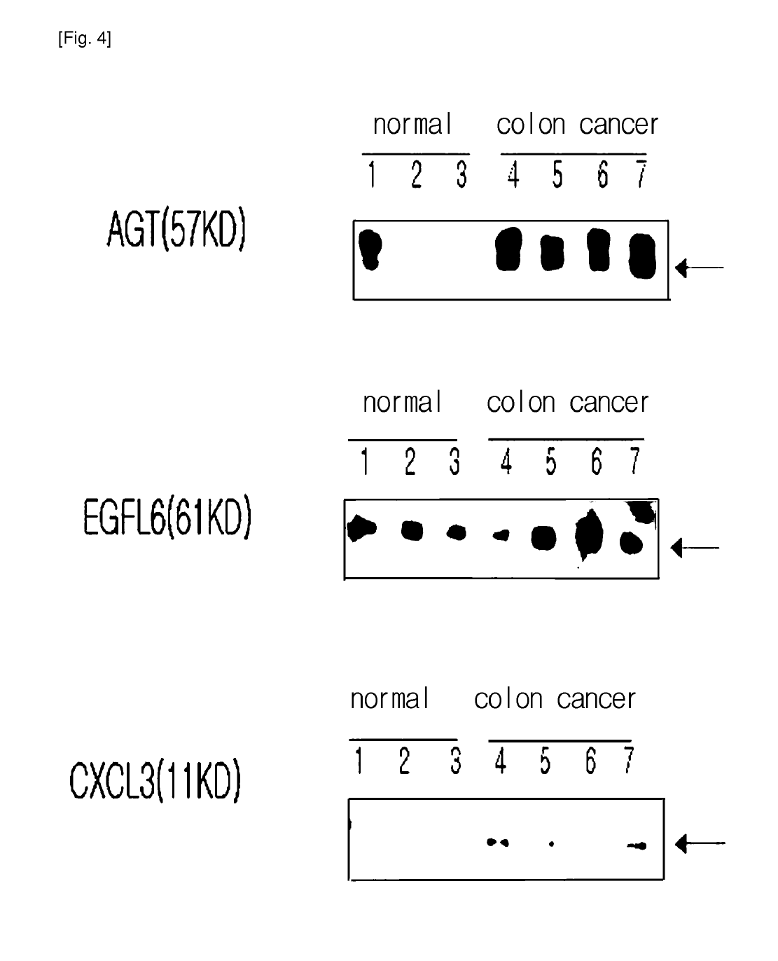

The results are shown in FIG. 4. AGT, EGFL6, and CXCL-3 proteins were not or little detected in healthy persons (lanes 1 to 3) while being overexpressed in patients with colon cancer (lanes 4 to 7), demonstrating the usefulness thereof as colon cancer diagnosis markers (FIG. 4)

### EXAMPLE 5

### Comparison of Protein Expression Levels in Tissues Using ImmunoStaining

Tissue slides were immunostained so as to determine the presence and expression positions of the proteins in normal colonic epithelial tissues and colon cancer tissues.

To this end, first, normal colonic epithelial cell tissues and colon cancer cell tissues were surgically excised from colon caner patients and embedded in paraffin blocks. Using a microtome, these blocks were cut into slices of 5 μm thickness, followed by the attachment of the slices to glass slides. The tissue slides thus obtained were immunostained, and observed for the presence and positions of the proteins in tissues under a microscope. The antibodies used in this immunostaining were anti-EGFL-6-antibody (Santa Cruz, 1:2000), anti-CTHRC1-antibody (SANTA CRUZ, 1:1000), and anti-CXCL-3-antibody (Aviva, 1:2000), and anti-AGT-antibody (R&D, 1:2000).

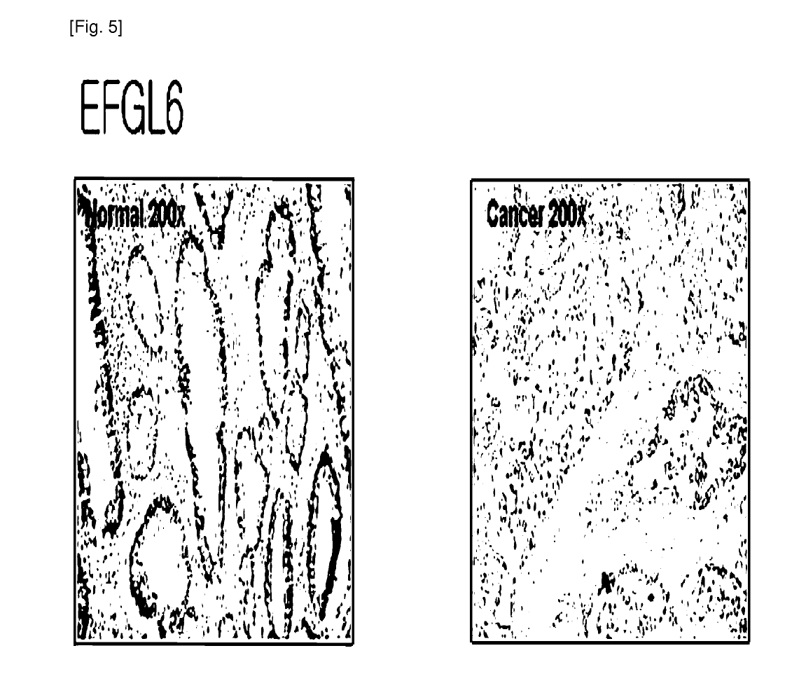

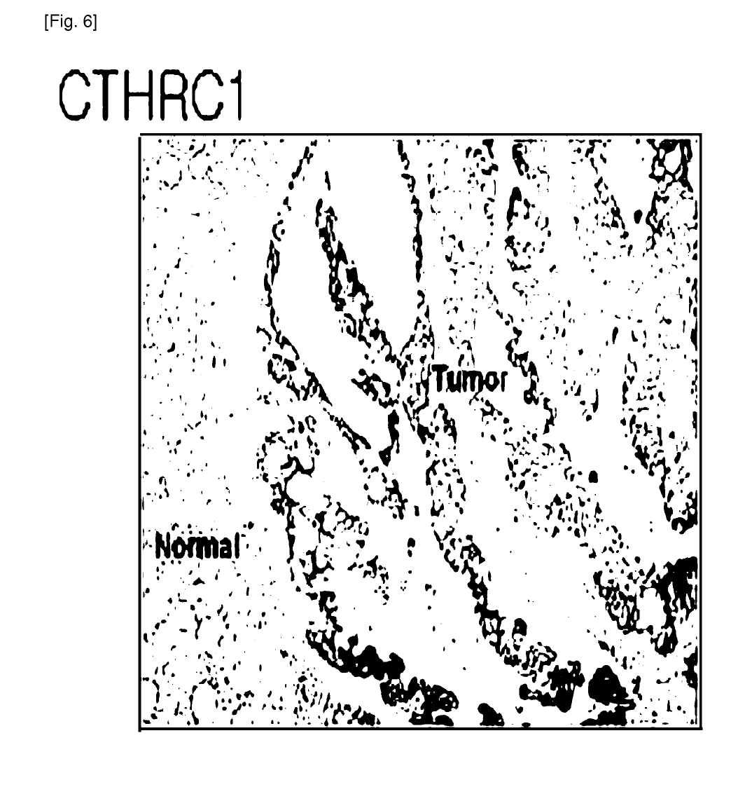

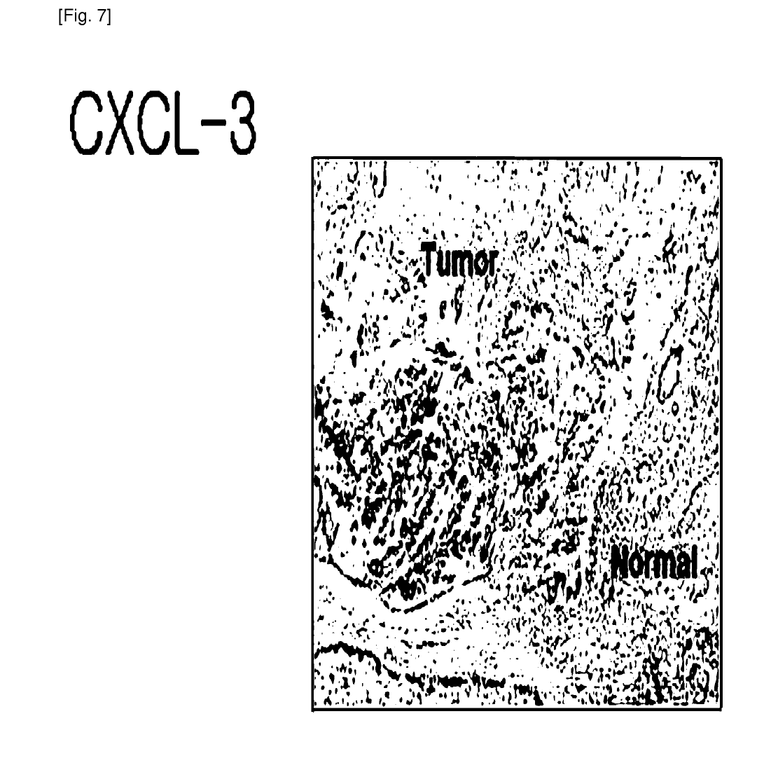

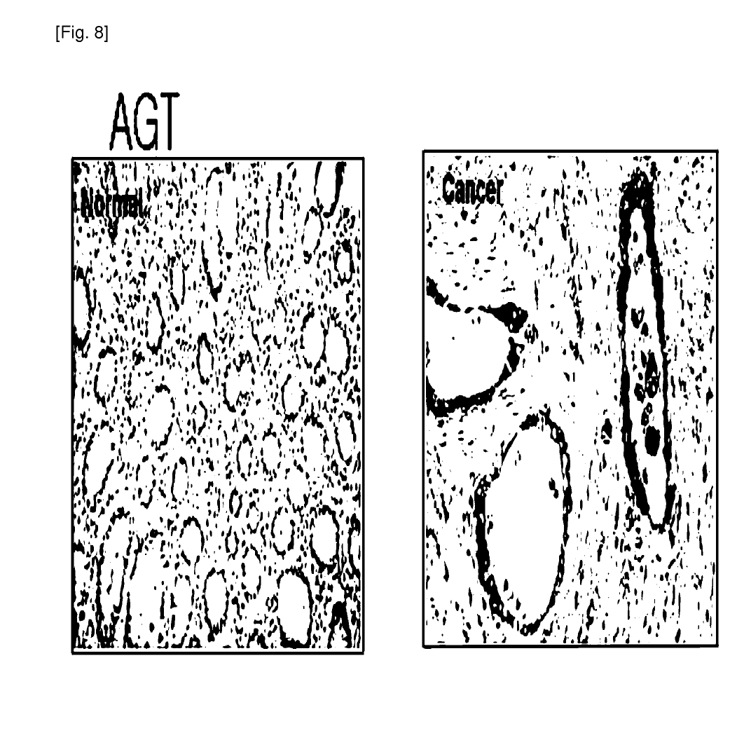

As a result, it was confirmed by immunohistological staining that EGFL6 and CTHRC1 were expressed in cell membrane and cytoplasm and more strongly expressed in colon cancer suspected tissues than in normal mucous membrane. CXCL3 was detected in cytoplasm or nucleus of tumor cells and AGT was expressed in cytoplasm and cell membrane of tumor cells and in endothelial cells as well (FIGS. 5 to 8).

### EXAMPLE 6

### Measurement of Protein Levels in Sera by Immunodot Analysis

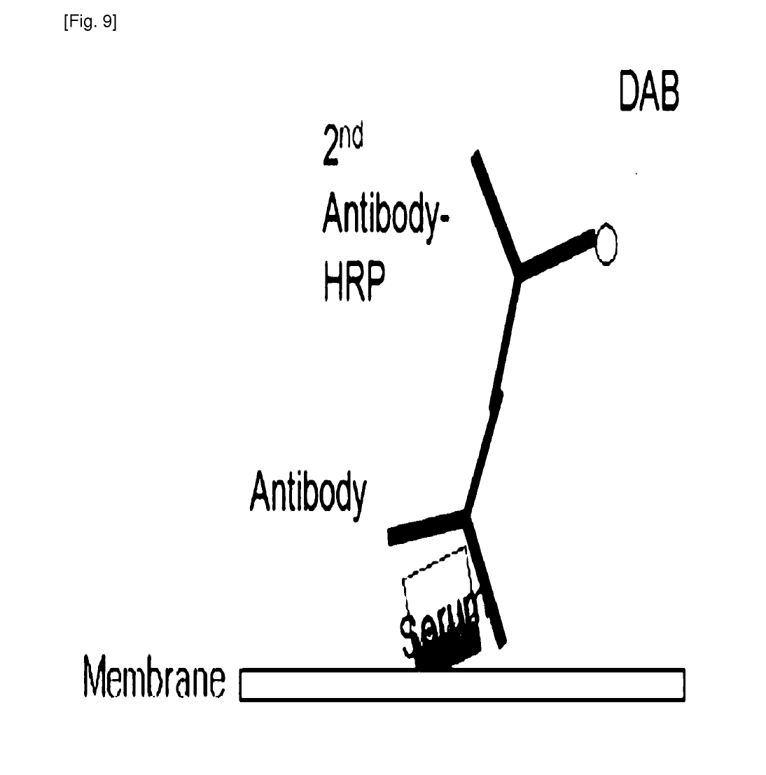

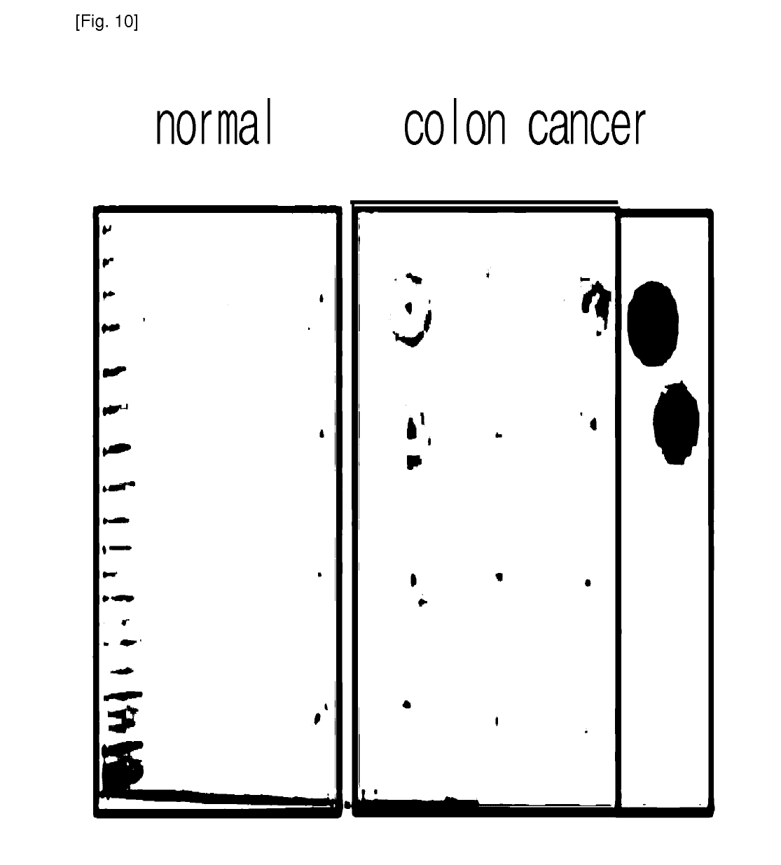

Sera from healthy persons and colon cancer patients were compared for secretion levels of the proteins AGT, EFGL6, CXCL3, COL5A2, CTHRC1, and FCGR3A using an immunodot assay with polyclonal antibodies. Each of the serum samples (10 samples per person) of a 5 to 10-fold dilution was dotted in an amount of 2 μl on a nitrocellulose membrane, dried at room temperature, and blocked in 1% BSAT (bovine serum albumin in Tris-buffered saline) solution. They were treated with a polyclonal antibody to AGT (R& D, 1:5000), a polyclonal antibody to EFGL6 (SantaCruz, 1:5000), a polyclonal antibody to CXCL3 (Aviva, 1:5000), a polyclonal antibody to Col5A2 (1:5000), a polyclonal antibody to CTHRC1 (SantaCruz, 1:1000), and a polyclonal antibody to FCGR3A (1:5000) and then with a horse radish peroxidase-conjugated secondary antibody (1:10000), followed by developing in a DAB solution (0.5 mg/ml, diaminobenzidine in PBT). Fluorescence data obtained by scanning were analyzed (FIGS. 9 and 10).

It was found to be expressed in larger amounts in colon cancer sera than in normal sera, demonstrating that these genes can be used as effective markers for the diagnosis and prognosis of colon cancer.

### EXAMPLE 7

### Establishment of ELISA System and Diagnosis of Colon-Cancer Thereby

7-1. Establishment of ELISA System

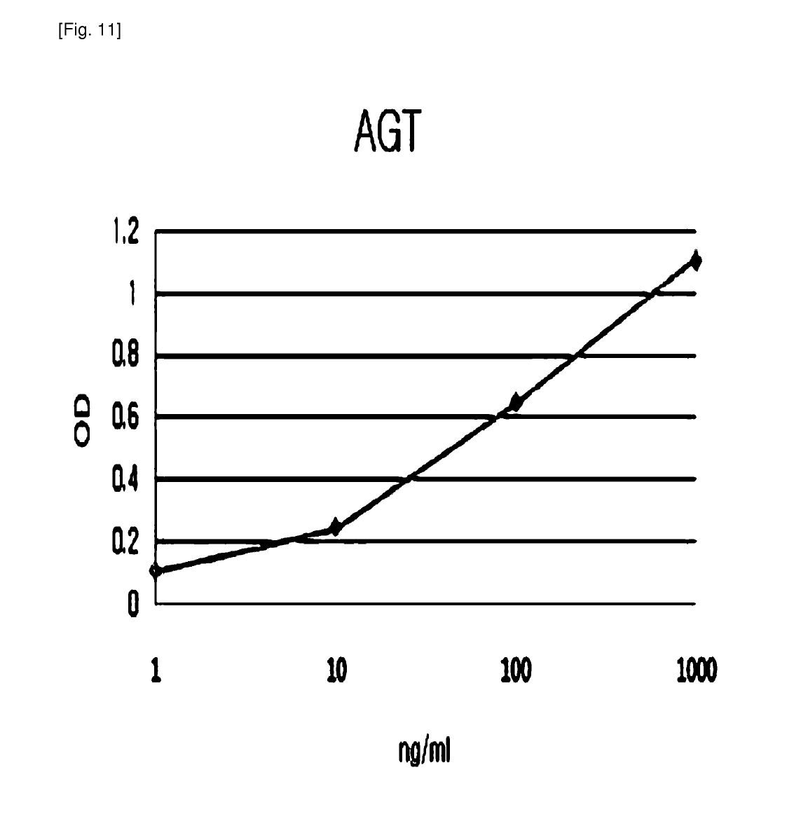

Monoclonal antibodies to AZGP1, EGFL6, CTHRC1, CXCL3, CXCL-6, AGT, FCGR3A, Col5A2, and S100P proteins were diluted to a concentration of 1 μl/ml in 0.1M carbonate buffer (pH 9.6) and plated in an amount of 100 μl/well into 96-well microtiter plates. After incubating overnight at 4° C., the microtiter plates thus coated with the monoclonal antibodies were washed three times with 0.05% Tween-20-containing PBS (PBS-T). Blocking at room temperature for 2 hours with 1% BSA was followed by three rounds of washing with PBS-T. Each dilution of the proteins corresponding to SEQ ID NOS. 1 to 9 was added in an amount of 100 μl to the 96-well microtiter plates and incubated at room temperature for 2 hours, followed by washing three times with PBS-T. Polyclonal antibodies (1:2000 dilution) to AZGP1, EGFL6, CTHRC1, CXCL3, CXCL-6, AGT, FCGR3A, Col5A2, and S100P proteins were added in an amount of 100 μl to the 96-well microtiter plates, incubated for 2 hours and washed. 100 μl of a 200-fold diluted, horse radish peroxidase-conjugated secondary antibody was added, incubated at room temperature for 1 hour and washed three times, followed by color development with TMB. Absorbance at 450 nm was read in an ELISA reader (Molecular Device, Sunnyvale, Calif., USA) (FIG. 11).

7-2. Measurement of Protein Levels in sera Using ELISA System.

Using the ELISA system established in Example 7-1, serum samples were measured for levels of AZGP1, EGFL6, CTHRC1, CXCL3, CXCL-6, AGT, FCGR3A, Col5A2, and S100P proteins. After being diluted five folds, normal and colon cancer sera were calculated for concentrations of AZGP1, EGFL6, CTHRC1, CXCL3, CXCL-6, AGT, FCGR3A, Col5A2, and S100P proteins.

### EXAMPLE 8

### Kit Construction and Measurement of Protein Level in Serum

8-1. Sandwich ELISA Kit

A kit for measuring concentrations of AZGP1, EGFL6, CTHRC1, CXCL3, CXCL-6, AGT, FCGR3A, Col5A2, and S100P proteins was constructed using the following components:

A. Solid phase antibody: A microtiter plate with an antibody adsorbed thereto. It was constructed by plating polyclonal antibodies to AZGP1, EGFL6, CTHRC1, CXCL3, CXCL-6, AGT, FCGR3A, Col5A2, and S100P in an amount of 100 μl per well into a microtiter plate, followed by incubating overnight at 4° C. to adsorb albumin to the solid phase surface.

B. Detection antibody: Monoclonal antibodies to AZGP1, EGFL6, CTHRC1, CXCL3, CXCL-6, AGT, FCGR3A, Col5A2, and S100P

C. Enzyme-conjugated antibody: horse radish peroxidase (HRP)-conjugated secondary antibody

D. Serum dilution buffer

E. Substrate (TMB)

F. Washing solution: 0.05% Tween-containing PBS (PBS-T)

G. Standard solution: Standard solutions of AZGP1, EGFL6, CTHRC1, CXCL3, CXCL-6, AGT, FCGR3A, Col5A2, and S100P proteins.

Using the kit, dilutions of sera taken from patients were assayed for levels of AZGP1, EGFL6, CTHRC1, CXCL3, CXCL-6, AGT, FCGR3A, Col5A2, and S100P proteins as follows.

A suitable dilution of a serum sample in a diluent (D) was added in an amount of 100 μl per well to the solid phase antibody of the component A, and analyzed for concentrations of AZGP1, EGFL6, CTHRC1, CXCL3, CXCL-6, AGT, FCGR3A, Col5A2, and S100P proteins using the sandwich ELISA kit established with the components B, C and E in Example 8-1.

8-2. Immunochromatography Kit

8-2-1. Construction of Immunochromatographic Strip

1) Preparation of Ab-Gold Conjugate

An antibody was added in a concentration of 15 μg/ml to a colloidal gold particle solution and then incubated at room temperature for 2 hours with agitation. To this solution was added 1/10 volume of 10% BSA, followed by the incubation of the resulting 1% BSA solution for 1 hour. Centrifugation at 12,000 rpm for 40 min precipitated Ab-gold conjugates. The supernatant was discarded and the precipitates were washed with 2 mM borate buffer. This washing process was repeated three times further. Thereafter, 2 mM borate buffer containing 1% BSA was added in an amount of about 1/10 volume of the gold solution to give a suspension. Absorbance at 530 nm was measured using a UV spectrophotometer and dilution was performed to form an O.D. of 3.00.

2) Sample Pad

Provided for absorbing a sample. Made of a cellulose material. As long as it absorbs samples, any can be used as a material for the sample pad.

3) Glass Fiber (GF) Membrane

Pretreated with 20 mM borate buffer containing sucrose.

4) Nitrocellulose (NC) Membrane and Line Treatment

A nitrocellulose membrane (Millipore) was cut into a suitable size (0.7 cm×5 cm). In the cut membranes, goat anti-sheep IgG was applied at a virtual control line about 3.4 m distant from the bottom of the plastic backing while monoclonal antibodies to AZGP1, EGFL6, CTHRC1, CXCL3, CXCL-6, AGT, FCGR3A, Col5A2, and S100P proteins were applied to a virtual detection line 2.7 cm distant from the bottom.

5) Absorbent Pad

Made of a cellulose membrane which can absorb materials remaining untreated after the immune response and thus allows the sample solution including analysates to migrate by capillary action.

6) Adhesive Plastic Backing

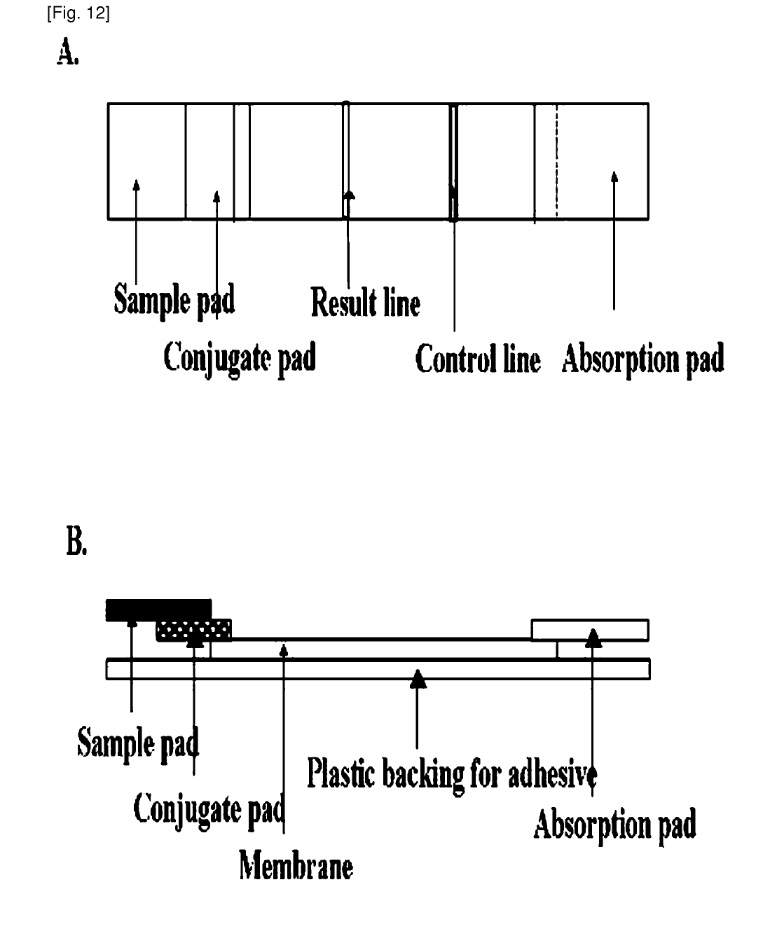

On an adhesive plastic backing, the sample pad, the GF membrane, the NC membrane and the absorbent pad were laminated, as shown in FIG. 12, in such a manner as for samples to continuously migrate by capillary action, thus affording an immunochromatographic strip.

8-2-2. Result Decision

3˜5 min after 6˜70 μl of a sample (e.g., a mixture of 1:5 (v/v) serum:elution buffer) was loaded on the sample pad, the strip was observed for color development at the control line and the result line and the concentration of the developed color. A positive sample developed red colors at both the control line and the result line. Only the control line was visualized as red for a negative sample.

8-3 Luminex Kit

8-3-1. Construction of Luminex Kit

Polyclonal antibodies to AZGP1, EGFL6, CTHRC1, CXCL3, CXCL-6, AGT, FCGR3A, Col5A2, and S100P proteins were conjugated to beads. A sample dilution was added in an amount of 100 μl and incubated at room temperature for 2 hours, followed by washing three times with PBS-T. Then, they were incubated for 2 hours with 100 μl of each of monoclonal antibodies to the proteins corresponding to SEQ ID NOS. 1 to 9 and washed. An additional one round of incubation was conducted at room temperature for 1 hour with 100 μl of a 2000-fold diluted, PE (phycoerythrin)-conjugated secondary antibody. They were washed three times before measurement in a luminex device. The fluorescence intensities were plotted against concentrations to give a standard curve.

8-3-2. Sandwich Luminex Kit

A luminex kit for measuring concentrations of AZGP1, EGFL6, CTHRC1, CXCL3, CXCL-6, AGT, FCGR3A, Col5A2, and S100P proteins were constructed using the following components.

A. Solid phase antibody: fluorescent beads with polyclonal antibodies to AZGP1, EGFL6, CTHRC1, CXCL3, CXCL-6, AGT, FCGR3A, Col5A2, and S100P proteins adsorbed thereto.

B. Detection antibody: monoclonal antibodies to AZGP1, EGFL6, CTHRC1, CXCL3, CXCL-6, AGT, FCGR3A, Col5A2, and S100P

C. Enzyme-conjugated antibody: peroxidase-conjugated secondary antibody

D. Serum dilution buffer

F. Washing solution: 0.05% Tween-containing PBS (PBS-T)

G. Standard solution: Standard solutions of AZGP1, EGFL6, CTHRC1, CXCL3, CXCL-6, AGT, FCGR3A, Col5A2, and S100P proteins.

Using the kit, dilutions of sera taken from patients were assayed for proteins as follows. A suitable dilution of a serum sample in a diluent (D) was added in an amount of 100 μl per well to the solid phase antibody of the component A, and analyzed for concentrations of AZGP1, EGFL6, CTHRC1, CXCL3, CXCL-6, AGT, FCGR3A, Col5A2, and S100P proteins using the components B, C and E.

8-4. Protein Microarray Kit

8-4-1. Protein Microarray System

Well chips from Proteagen were coated with monoclonal antibodies to AZGP1, EGFL6, CTHRC1, CXCL3, CXCL-6, AGT, FCGR3A, Col5A2, and S100P proteins. The chips were blocked with BSA buffer, incubated at room temperature for 1 hour with 100 μl of a serum dilution, and washed three times with PBS-T. Again, the chips were incubated at 37° C. for 1 hour with 100 μl of each of diluted monoclonal antibodies to AZGP1, EGFL6, CTHRC1, CXCL3, CXCL-6, AGT, FCGR3A, Col5A2, and S100P proteins and washed. An additional one round of incubation was also conducted at room temperature for 0.5 hours with 100 μl of a 2000-fold diluted, Cy3-conjugated secondary antibody. The chips were washed three times before the measurement of fluorescent intensity at 532 nm. The fluorescent intensities were plotted against concentrations to give a standard curve. The protein microarray system thus established was used to determine the serum levels of AZGP1, EGFL6, CTHRC1, CXCL3, CXCL-6, AGT, FCGR3A, Col5A2, and S100P proteins.

8-4-2. Sandwich Protein Microarray Kit

A sandwich protein microarray kit for measuring concentrations of AZGP1, EGFL6, CTHRC1, CXCL3, CXCL-6, AGT, FCGR3A, Col5A2, and S100P proteins were constructed using the following components.

A. Solid phase antibody: a slide coated with polyclonal antibodies to AZGP1, EGFL6, CTHRC1, CXCL3, CXCL-6, AGT, FCGR3A, Col5A2, and S100P proteins.

B. Detection antibody: monoclonal antibodies to AZGP1, EGFL6, CTHRC1, CXCL3, CXCL-6, AGT, FCGR3A, Col5A2, and S100P

C. Enzyme-conjugated antibody: Cy3-conjugated secondary antibody

D. Serum dilution buffer

F. Washing solution: 0.05% Tween-containing PBS (PBS-T)

G. Standard solution: Standard solutions of AZGP1, EGFL6, CTHRC1, CXCL3, CXCL-6, AGT, FCGR3A, Col5A2, and S100P proteins.

Using the kit, dilutions of sera taken from colon cancer patients were assayed for proteins as follows. A suitable dilution of a serum sample in a diluent (D) was added in an amount of 100 μl per well to the solid phase antibody of the component A, and analyzed for concentrations of AZGP1, EGFL6, CTHRC1, CXCL3, CXCL-6, AGT, FCGR3A, Col5A2, and S100P proteins using the components B, C and E in the same manner as in the sandwich method of Example 8-4-1.

### INDUSTRIAL APPLICABILITY

As described hitherto, the present invention provides diagnostic markers for accurately diagnosing colon cancer at an early stage and determining the metastasis and prognosis of colon cancer, thus affording data useful in the treatment and monitoring of colon cancer.

With ability to determine mRNA or protein expression levels of genes specific to colon cancer readily and rapidly, the colon cancer diagnosis markers of the present invention can also be used in research for developing anticancer agents against colon cancer.

Although the preferred embodiment(s) of the present invention have(has) been disclosed for illustrative purposes, those skilled in the art will appreciate that various modifications, additions and substitutions are possible, without departing from the scope and spirit of the invention as disclosed in the accompanying claims.

1-17. (canceled)

18. A method of diagnosing colon cancer in a subject, comprising:
a) measuring a level of CXCL3 (C-X-C chemokine ligand 3) mRNA or protein in a biological sample from the subject; and
b) determining the presence of colon cancer in the subject, wherein an increase in the level of CXCL3 mRNA or protein as compared to a normal control subject indicates the presence of colon cancer.

19. The method according to claim 18, wherein the biological sample is selected from the group consisting of tissue, cell, whole blood, serum, plasma, saliva, sputum, cerebrospinal fluid and urine.

20. The method according to claim 18, wherein the level of CXCL3 mRNA is measured by a RT-PCR (reverse transcription-polymerase chain reaction), Competitive RT-PCR, Real-Time RT-PCR, RPA (RNase protection assay), or Northern blotting.

21. The method according to claim 18, wherein the level of CXCL3 mRNA is measured by using a primer set comprising a forward primer of SEQ. ID. NO: 12 and a reverse primer of SEQ. ID. NO: 13.

22. The method according to claim 18, wherein the level of CXCL3 protein is measured by an immunodot assay, a luminex assay, an ELISA assay, a protein microarray assay, an immunochromatographic strip assay, or western blot assay.

23. The method according to claim 18, wherein the level of CXCL3 protein is measured by using an antibody specific to the protein.

24. A method of diagnosing colon cancer in a subject, comprising:
a) measuring levels of CXCL3 mRNA and one or more additional mRNAs in a biological sample from the subject, wherein the additional mRNAs are selected from the group consisting of AZGP1 (alpha-2-glycoprotein 1, zinc-binding) mRNA, CXCL6 [chemokine (C-X-C motif) ligand 6, granulocyte chemotactic protein 2] mRNA, AGT [angiotensinogen(serpin peptidase inhibitor, clade A, member 8)] mRNA, FCGR3A (Fc fragment of IgG, low affinity Ma, receptor) mRNA, Col5A2 (collagen, type V, alpha 2) mRNA, S100P (S 100 calcium binding protein P) mRNA, EGFL6 (EGF-like-domain, multiple 6) mRNA, and CTHRC 1 (collagen triple helix repeat containing 1) mRNA; and
b) determining the presence of colon cancer in the subject, wherein an increase in the levels of CXCL3 mRNA and one or more additional mRNAs as compared to a normal control subject indicates the presence of colon cancer.

25. The method according to claim 24, wherein the biological sample is selected from the group consisting of tissue, cell, whole blood, serum, plasma, saliva, sputum, cerebrospinal fluid and urine.

26. The method according to claim 24, wherein the levels of mRNAs are measured by a RT-PCR (reverse transcription-polymerase chain reaction), Competitive RT-PCR, Real-Time RT-PCR, RPA (RNase protection assay), or Northern blotting.

27. The method according to claim 24, wherein the levels of mRNAs are measured by using primer sets selected from the group consisting of the primer sets of following 1)-9):
1) SEQ. ID. NO: 12 (forward) and SEQ. ID. NO: 13 (reverse) for CXCL3;
2) SEQ. ID. NO: 10 (forward) and SEQ. ID. NO: 11 (reverse) for AZGP1;
3) SEQ. ID. NO: 14 (forward) and SEQ. ID. NO: 15 (reverse) for CXCL6;
4) SEQ. ID. NO: 16 (forward) and SEQ. ID. NO: 17 (reverse) for AGT;
5) SEQ. ID. NO: 18 (forward) and SEQ. ID. NO: 19 (reverse) for FCGR3A;
6) SEQ. ID. NO: 20 (forward) and SEQ. ID. NO: 21 (reverse) for Col5A2;
7) SEQ. ID. NO: 22 (forward) and SEQ. ID. NO: 23 (reverse) for S100P;
8) SEQ. ID. NO: 24 (forward) and SEQ. ID. NO: 25 (reverse) for EGFL6; and
9) SEQ. ID. NO: 26 (forward) and SEQ. ID. NO: 27 (reverse) for CTHRC1.

28. A method of diagnosing colon cancer in a subject, comprising:
a) measuring levels of CXCL3 protein and one or more additional proteins in a biological sample from the subject, wherein the additional proteins are selected from the group consisting of AZGP1 (alpha-2-glycoprotein 1, zinc-binding) protein, CXCL6 [chemokine (C-X-C motif) ligand 6, granulocyte chemotactic protein 2] protein, AGT [angiotensinogen(serpin peptidase inhibitor, clade A, member 8)] protein, FCGR3A (Fc fragment of IgG, low affinity Ma, receptor) protein, Col5A2 (collagen, type V, alpha 2) protein, S100P (S 100 calcium binding protein P) protein, EGFL6 (EGF-like-domain, multiple 6) protein, and CTHRC 1 (collagen triple helix repeat containing 1) protein; and
b) determining the presence of colon cancer in the subject, wherein an increase in the levels of CXCL3 protein and one or more additional proteins as compared to a normal control subject indicates the presence of colon cancer,

29. The method according to claim 28, wherein the biological sample is selected from the group consisting of tissue, cell, whole blood, serum, plasma, saliva, sputum, cerebrospinal fluid and urine.

30. The method according to claim 28, wherein the levels of proteins are measured by an immunodot assay, a luminex assay, an ELISA assay, a protein microarray assay, an immunochromatographic strip assay, or western blot assay.

31. The method according to claim 28, wherein the levels of proteins are measured by using antibodies specific to the proteins.


SEQUENCE LISTING (29 sequences)

SEQ ID NO: 1 - DNA, 1247 bp, Homo sapiens
gataatatctgtgcctcctgcccagaaccctccaagcagacacaatggtaagaatggtgcctgtcctgctgtctctgctgctgcttctgggtcctgctgtcccccaggagaaccaagatggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacatcgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataaaaaaaaaaaaaaaaaaaaaaaaaaaaaaaa

SEQ ID NO: 2 - DNA, 1166 bp, Homo sapiens
gctccgggaatttccctggcccggccgctccgggctttccagtctcaaccatgcataaaaagggttcgccgatcttggggagccacacagcccgggtcgcaggcacctccccgccagctctcccgcttctcgcacagcttcccgacgcgtctgctgagccccatggcccacgccacgctctccgccgcccccagcaatccccggctcctgcgggtggcgctgctgctcctgctcctggtggccgccagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactgacaggagagaagtaagaagcttatcagcgtatcattgacacttcctgcagggtggtccctgcccttaccagagctgaaaatgaaaaagagaacagcagctttctagggacagctggaaaggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtggggggattgaaatgcaagcaattagtggatcactgttagggtaagggaatgtatgtacacatctattttttatactttttttttaaaaaaagaatgtcagttgttatttattcaaattatctcacattatgtgttcaacatttttatgctgaagtttcccttagacattttatgtcttgcttgtagggcataatgccttgtttaatgtccattctgcagcgtttctctttcccttggaaaagagaatttatcattactgttacatttgtacaaatgacatgataataaaagttttatgaaaaaaaaaaaaaaa

SEQ ID NO: 3 - DNA, 1677 bp, Homo sapiens
accccttctttccacactgccccctgagttcagggaatttccccagcatcccaaagcttgagtttcctgccagtcgggagggatgaatgcagataaagggagtgcagaaggcacgaggaaaccaaagtgctctgtatcctccagtctccgcgcctccacccagctcaggaacccgcgaaccctctcttgaccactatgagcctcccgtccagccgcgcggcccgtgtcccgggtccttcgggctccttgtgcgcgctgctcgcgctgctgctcctgctgacgccgccggggcccctcgccagcgctggtcctgtctctgctgtgctgacagagctgcgttgcacttgtttacgcgttacgctgagagtaaaccccaaaacgattggtaaactgcaggtgttccccgcaggcccgcagtgctccaaggtggaagtggtagcctccctgaagaacgggaagcaagtttgtctggacccggaagccccttttctaaagaaagtcatccagaaaattttggacagtggaaacaagaaaaactgagtaacaaaaaagaccatgcatcataaaattgcccagtcttcagcggagcagttttctggagatccctggacccagtaagaataagaaggaagggttggtttttttccattttctacatggattccctactttgaagagtgtgggggaaagcctacgcttctccctgaagtttacagctcagctaatgaagtactaatatagtatttccactatttactgttattttacctgataagttattgaaccctttggcaattgaccatattgtgagcaaagaatcactggttattagtctttcaatgaatattgaattgaagataactattgtatttctatcatacattccttaaagtcttaccgaaaaggctgtggatttcgtatggaaataatgttttattagtgtgctgttgagggaggtatcctgttgttcttactcactcttctcataaaataggaaatattttagttctgtttcttggggaatatgttactctttaccctaggatgctatttaagttgtactgtattagaacactgggtgtgtcataccgttatctgtgcagaatatatttccttattcagaatttctaaaaatttaagttctgtaagggctaatatattctcttcctatggttttagacgtttgatgtcttcttagtatggcataatgtcatgatttactcattaaactttgattttgtatgctattttttcactataggatgactataattctggtcactaaatatacactttagatagatgaagaagcccaaaaacagataaattcctgattgctaatttacatagaaatgtattctcttggttttttaaataaaagcaaaattaacaatgatctgtgctctgaaagttttgaaaatatatttgaacaatttgaatataaattcatcatttagtcctcaaaatatatatagcattgctaagattttcagatatctattgtggatcttttaaaggttttgaccattttgttatgaggaattatacatgtatcacattcactatattaaaattgcacttttattttttcctgtgtgtcatgttggtttttggtacttgtattgtcatttggagaaacaataaaagatttctaaaccaaaaaaaaaaaaaaaaaaa

SEQ ID NO: 4 - DNA, 15628 bp, Homo sapiens
tttaaagccttacgtagaagatcccccagctgatagtcagccttgggcatggattaagggcttttaaccaatcttgcaacaagtttaagcagatattctttattgggtccaatctaaccaaaattattttcttatgttctccccagtaacgtgtcattattaagagaagtttggcttgcttagaggccaaatttagagggtcctgaaattttattttcttttacaccactttccagcatgttacctgatcagttgtttattatctttgctgttgaatggagtgatcattccaagggcccgaggcaggaggcccaggcacagtggaaactctcccaaagaccaggatctttgttttgttccctgacatatgctgagcaccaggaatagtgaatgaatgaaacaaattgtgaggctttaaagagccgaaatatttaaacactgggcacaaggttgttgcttaatcagtgctagatccttacctcccccttgtgtccaggtcgacttgttactgcagttaaaccacttgctgatcctcaaacaactagttagtggcacagccaggcctaggaccccagtctctactgttccaactaacccattcgcaggcaggagcactttgaatggtctcttattttaaaaaaattaaattaaaattgtctatttatttagagacagagtcttactctgtagcccaggctcgagtgcagtggtgcaatcatagctcactgtaacctccatctcctggcctcaaaaagtgtttgaattacagatgcgaggcactgtacctggcccgaatgttctgttcagacaaagccacctctaagtcgctgtggggccccagacaagtgatttttgaggagtccctatctataggaacaaagtaattaaaaaaatgtatttcagaatttacaggcccatgtgagatatgatttttttaaatgaagatttagagtaatgggtaaaaaagaggtatttgtgtgtttgttgattgttcagtcagtgaatgtacagcttctgcctcatatccaggcaccatctcttcctgctctttgttgttaaatgttccattcctgggtaatttcatgtctgccatcgtggatatgccgtggctccttgaacctgcttgtgttgaagcaggatcttccttcctgtcccttcagtgccctaataccatgtatttaaggctggacacatcaccactcccaacctgcctcacccactgcgtcacttgtgatcactggcttctggcgactctcaccaaggtctctgtcatgccctgttataatgactacaaaagcaagtcttacctataggaaaataagaattataacccttttactggtcatgtgaaacttaccatttgcaatttgtacagcataaacacagaacagcacatctttcaatgcctgcatcctgaaggcattttgtttgtgtctttcaatctggctgtgctattgttggtgtttaacagtctccccagctacactggaaacttccagaaggcacttttcacttgcttgtgtgttttccccagtgtctattagaggcctttgcacagggtaggctctttggagcagctgaaggtcacacatcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcatcgtgacccggccaggggaagaagctgccgttgttctgggtactacagcagaaggtaagccgggggccccctcagctccttctcggccttgtctctctcagatgtaactgagctgtgggctaggaggaaaaggccgggaggaggcacggtgatgactgaaaaacctctcccctctcataagaccagtcatccggacgcgggctttcccccactcggtgcccacctggggtcttacaggaggagctgctcctcctcagcaataggacaagatggtcaggtcttcctgcttccgctgagaaaagttagggtcctcaggaacggagcagactggtacaggaacagagtcatcatggccaagagtccaccgggtcctcttgccatcaggaggaatagcagggcttgtgcaggaattggggctggagggaagggccgggctcggtcagtctccagctgggatccccagagtggtcaccctacccctccctcgagacagactgcctgactgtgtgtcatcaggctggtcaccgtctccctgaacctcgatttgctcacctataaaatggaactaataacgatgcctgggctccctgtctcaggggctctggtatagctgaagagaactaatataacatgaaagtgctttctaagctttgggataagctaaaaggcagattccaattttattcgagggcagcgtagattggtgcttcagctcgtggatgacagagtcagggggcctggttctgagtcctagttctgtctcttcccagctgtgtgacgttgaacaagtcactggacctctctgttcctctgcaaaacagcatgaaccaattcattaactacttctccaggatgcagtaggtcccagggactatcctaggaatgtgggctgtattagtaaacacaacagcgggaaccctgttccggggctcacattcacatcagagcaaacagacaaagacgctggacagaataagtgcataactacatggtacagagggttataaggagggaaaaggggagctggatgagagagttgagagtgcccggtgtggtggggaaagctgcagggtgaaatactgcatcagggaaacctcagggaaggtgaggactatggtgaggtcagaggggttgatatgagaacagtgccctgcaaatggcaggcaccacaggagcatgagccgtcatcttcacctttagcattcagcccgggagaagtagggagacatagaaggggcaggtgctggccaagaggcaggggcaggagaggagaaggcggaggggcactcagggcgagggtgtcaggcccgccaccccagagcaccattactcccaggacgcggctgcgtgcagacctggaaccagcctagggagcagccgcagatcacaactgagaacaaacgacagtctctgcctcaaaaatggcccatggaattgcgtctctggagacgctgcctgagcaggagcagcacagtgagcgggctgcatcgaccagcgccatccaaaccccgaacagttggcgcttgtcaggcaggacttcccagcagtcggttcccacaggtttcccctgttgacctgatttgatgtgactgtctagattaggtgtgaactggtggcttaggcttctctgcacagaaaggcctgcaagcagcagagagagttttctgttccatttttccatgtcatgtggctcttcctgagaacagcggatggagtcaaatgcatggggagtggggtgagatggtagctgaggtcagaatttggcatttgaatgactgaagcagaacaaaacacaccaggtacttcagcagctgcaccgtgttgagggcaggtgctggttacgggtctgggtgagggaagccagctgccaatgtaagaagaatgactgggtatgcttagatgaagcagaaaaatctaggcatcaaggtggccttgagtcagtgatgacacgctacagctccaaggaagcctggcctagccctggggggacagaaaaggccaagaagtgacgatattgcagtacacccccctccacaagaaatgagtgagatgtggtacaaaatgttagaattgaatgaatcaatagaataaacgttcatcccttcaatcaagaagagtcagatgaaatgaattagcagggccagcccaagaacctcttctgggggtctcagggtagctttcatttgtagcagctgaggctgaagcccagctgcaaggcctttgagagaacgtggtgctggacccgtgtctagggcaggggttctaaaccctgcttacatatcagagtcacctgagaattttctatttttttttttttttttttatacgtggtcccagcacagactaaggaatccaactatcattgggcaagccatgctaggtatgcatgcctttggggctctgcaggggatagcgctatgcagggatggttgagagctggttttggggttgagacacgtgggaaatacttggactttgggctgagcctgtggtgctcaatcccggctgcatgttgggaccacagggagatgacaaaaccatccccagccctcaccctagggccctcgaatgagcatctcaggggtctaggaggcctccacaaagacctactgattggcacacacttgtttctctaggaagagaacttacagctgcaggcaggagcatgtcttaatctgcttgggctgccataagtaccacagactgggagggtttaacaacagaaatgtgttatctcacagttctggaagctagaagcctgggagccagccatcagcagagttggtttcctctgggtcctctatccttggcttgtagatggccgtcttctctctgtgtccccacatggtcttccctctgtgtccccacatggtcttccctctgtgtgtgtccatgtcctcatctcctcttctcataaggacacaggtcatattagatcagggctcaccctcatggcctcattttaacttaatcatctctttaaagatcctgtctccaaataatggtcacattctgaggtcctggggttgaggacttcaacacgggcattatggccgttgggggaggtaggacataattcagctgatattggtgcattttgcacttggatcatgtagatattttccatggagctttgaatccatttcttcttttttttgtagacatgaatggatttattctgggctaaatggtgacagggaatattgagacaatgaaagatctggttagatggcacttaaaggtcagttaataaccacctttcaccctttgcaaaatgatatttcagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgacgggagccagtgtggacagcaccctggctttcaacacctacgtccacttccaaggtaaggcaaacctctctgctggctctggccctaggacttagtatccaatgtgtagctgagatcagccagtcaggccttggagatgggcagggggcagccctgcggacatacctggtgaccacccttgagaagtggggaagtggctgctccgctgggtccctggatgggccgtccacctcctggacctgctgccctactatgtgcacgactatacaacatcctttttcttacatcatttaatccccttatgatgtggtgaagaggtatttgtgcctttgtttaccagtgaagaaatagagactcggagaaacaaagtgccttgctcaagatggcacagccaccagtgggggtcctgggattgaaacccacatctcctggccccacagcccagttctacactcagaagggtcaggttcatatctcttgagaaggtcaggaactggggtccctggcccatgcagaaataagcaattggcttgcttaaatccctttcatgttaggaggggcattactgaaaaccctctactacaaagattgttgatttttttttttttttttattgagacagggtcttgttctgtcacccaggctgcagtgtagtggtgccatcattgctcactgtagccttgaactcctggcctcaagcgatcctcccacctctgccttccaaagtgttgggattaaaggtgtgagccactgcacccagccacagattgcttaaagcattcatttaacaaatacttgttgaggatttgctacttgtaagactttaagcctggcatctcagaggaggccagaggagggctgtataggccctgcctccaggcttttaaaggtcaatgggcaaatgcctaggatttggagctgcagggaaacgtgctccacaaggtaactcagggaagcctcggggctctcagaggacagaggtcactggggagcggagagcaggccttgcctggcagtgagggcaacagggctggtgaagctaggagcaagcatgatgagcccagcctgcagagtttggggcaaggaacgaggatggggcggttggcttggcatgagtgttgaaccagaaaatgggcctggggagggcagagctggagacactttgaacgccatgcttggtaggtgtgggaatggggacgcgttctgttcagaggtcatcccggaagcctgccgtgtgcagactggaggcagggaggattgtttgaaggttacgcaagagtccaggcacacagtcacgggaacacgtgctcagggagcagctcggcaaatccatgggtggggtggggctgaggggtgtgtctaagagacactgaggaggctctgtcaagatgttaacctcgtgagggacagagagccaggcgggaggtgaaagacaagactgtggagaaagaggttcagtggcgcatagtgatttttcttaccacaacaacctccttgaggtctttcccttcgggttcagggagaggtgatagatggggggattgctcagccctggcactgactggtcacaggggcagaggccagcccgagggttgcccggttgagggtggcagcacactgtgcagggcagagcagggacacatggacttagcctgctgtccctaggagaagtgctgggaggagcgctcactgagaaggagggtcctgcagaaggcaaaggcaagaaagccagtggcatctgaaatgggtctcccttcgaaagagagcacatccacctgacccagaccgcagagccaggccaggaggaagaggaggaagaataaaaaagccaaccacatcgggactcaaaggaagcccaggatcctcgccggcctccaccgcatgctgccctgaccctgccccacttcctaactttgctggcctcagtttccgtcaaaggaggcagccacttcctgcccacatggtctgtccagtgaggagatcgggggctgtctcgggacctctaggtttccctttagcaatgatgttctatttacatgacctcagcaggcagctagatgtgtcccactagagaggacctgaggatctggggcctgatgggctccagggtaccgtctgcccagtgcttgctgtgctcctgagcatggggcgctggccctggtggtttccatgacaccaggtcctgacttgacctcgacagatttacctagcctccggatgagaatggtgagctgtgcatgtcagacgagcagagggaagacggcagccactctcatgtcaaatcccagcgtcttttgggaggcagcttcccttttttagtttagtttgttggaagaaaagaattgtccctttcccccctctaaactaaaagccttgccagcccaggtgggcagcaccgaggtccctgcagggaacgtgcaaggggaaccctgcagtttcccgctcacatgcccttccgagactgagtgctccgaggactgaggacgagaaatatgccaggtctgccactgccttcttacgagacccggacccaggggaggcacagccatgcccagctcctgcctgccagttctgtcctcccagctgccctactttcatgctgggacctccaattcagtacaaagggagacctcactgtttctgaaccatctctactcagactcccaagtgccacgtgcccaggggactgttctgtgacaaacttatacacaacttcaccctattctcctaagaacaaccgcagaataggcctttcaggatgagtgggaggacagccgagggcagggatgtgctagtgtaaggtcgaggcagagggtgggctgctgtcatggaaagaccccaggtaactgcgtcacacacaaatttgtgtccttctcccacaacgggctctcccgagttctctgtcatctgcacggccctgtgagcaggaggggaaacagagggctcacccctgcccccaaggcccagtgtgcaaatccattcatcacaacgaggttgtgtgagtctccccagtagcaagggctgctgaggaatggagccctcgtttccggggcctgcgtggcccactctgtattctatgactgtgatgggggagggtgggggccacaggacagctggtgggctctgccatggctggggctagacatggattaaaaagtgagtatgagcaggggcctctaggagtggtgggatagtgcggtggtggccacatgtcattctacgtgcgtccaaacctacagaatgtaaaacaccaggagggagactcaaagaaaactatcaactttgagtgctgaggacgtgtcagtgtaggttcgtcagttgcaacaaatgggccacgctggtgtgagatgttgatcacgggggaggctgtgtagtgggggacaagagttatatgggaactttctgtactttctgctcgattttgctgtgaacctaaagtcactctaaaaaataacatctcttaaattttttaaaaagtgagtgtgtcaaaccacagcctttgggtcaggacagttctaggtttgagttgacctggcaggtaccagtggcttatgtcccttaaggtgacagatgcaaaacccccggtttggtgcctggcatgttgtgtgtcttgcaggtggcggttagggctgcctcagtgaactcaaatggctgcattttacaggagaaatatttgagccacacttgcggtcctgtggccaggagaatgcagagtggcctggggggggccaaggaaggaggctgaggcagggcgaggggcaggatctgggcctttggtgtctgccagccctcattcctgcccctgtcttgggtgactcttccctccctgtctcctgtctggatttcagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactgtctccccggtaggagcctcccggtctcccctggaatgtgggagccacactgtcctgcccaggctgggggcggggtggggagtagacacacctgagctgagccttgggtgcagagcagggcagggccgcggtggcacggggctgggcaggcggcctgtgtgtctgtctaccagtcctccatccagccagcacccagctctccagttagtgtctgtctttcaagtgcaggcaaggtaaaggaggagaggaagaatgctttttctacacttacacttgcctggtagttttggagggggagaaaacattgcaatccgccctctgagagaggaccattttggtcccacacctgacacacagcacacctgtgacatccaagagcttcttggaactgacttgccaggagggttcggacttcgcgtgagcgggggtggggccttctcagggagcgtcccttgactccagaacgcccttgctggcggctggcggctgggtggggataggtgttgttagctcctctttcctgctgcaattcctttccacagagccctggactcaaactacacatcaccccagatcatcgaggcctggaaatctgctcccagaggcaggcattgagtgacacgatggcttgacatcaactctgggtgttttttatgttttaaaaattgtgatggtaaaatatacgtaacaaaatttgccatcgtaaccattttcgagtgcacagttcagtggtactaggcccattcacactgttgtgcagccatcacccccgtccatctccatttatcttctcaacttcccaaactgaagctctgtcctgctgaaacactaactctccatttccccttccccttggccccggcaaccaccacgatgtcctcgaggttcacccatgttgtagcacatgtcagaatgtccttccttttgaaggctgaataatattccattgcatgtggttaccaccttttgtgtatccactcatccatcgatggacacgtgggttgcttccacctttgagctgctgtgaatagtgcagtgtaccctgtaaacatgggtgtactgtcagctcttataagtgcttgatacatcactggaaatgtccatgggctctgaaggatgccaaaagatggaagaggctctatacgaagatcaatcgagttgacatagcaacgtgtccagcacgaggttgacactgtaccctcctgcctctctccttttcatgggtgtcatgtcatcaagaacactgctgtggcagtagtaagacacagtgcattatttcagagaatagcatttaaaaattacccaagtaacacaccttcaatgcagccaacctaaaaacagaatgcaccaaaggacaaccattcctaggtcctcatcggtaaatcttctatgtccctcacatagtattgcaaatgacatgaaggatttttattgtaggttttgctgaaattttccccaagggggaggatgacttagttgggtgatggggggagcaaacatccctgtcgtcagggttgggtgcaaggagcataagcctgcctggcctctgggagagccctcactgtgtggcctggagccttcctaactgtgcatcatctccccaggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtatgtgtgagcctgtgtctgtgcctgacctgggttccaagtgtgcacagggtgggaggcatggatgtaagggacacagaggaggctatgggtggggccagcagggcaagagggagcggagagtagggccaaaggtgggagagaagtagccagagcattctggggccttccaggtgcagagcagcaaatccctccccatccctgctgtgcctcctcctgctaggtgtgtgttccatggtcctgcttggccttgccttgcctcagggtcctccagggttcctatagtggagttgaaaccgggatgaagacagcaagcacccctggacctggtgccctgggcccagccccttcttcagggaaatgctgagcagcagacagaatgtccccctgccatgtggcaccatgcacatctgcagctaccaaggatgtgccttgatgttctgggccctgtgctcagtgctggggagaaagtgggagttcttacgggggccagcgggaagagccctctgtgctaagttagctaagccctggcactggtgggccatggccaagggagccaggaattctgcctgggacatcagggcagaatgtgaagatgggaggatgtaaggggtgtgttagggaggagccggcatgtgagtttggccattgtggccaattaacggtcatctacacacagacacacccttgcctacactgaggggcaggcatacactgtgcatcctcctggcaggctggaaaatgtccccctccaggacagtgcacagcacagaggtcctgagcccaccccggccctctagccctcagcaccctgggtcacccagtgcgccctcagaatgatcctgatgtctgctgctttgcaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaattttatctttcatttagcagccaaacagatgtatacaattcagcagatagactgtgcaaacgaaagtgctttcctggactttggatggaatttccatgggaggtctgagccagtacttagcagtcctttgaagttttaggtgatgcttttctctggacacttccattggtaagcagtggtggccatctgtgtgatggacagggggcgggaagagggtgacagggaaggccccataccccatgtggcacctgggaaaggaaccaggcagatgggacttcttccgtcctggtgacacagggccagactgctgctggtattgtgccccgggagtggaaggtagagaaataaatcttcacaaataaatatttgcaattttcccccatctgttgagtgcctctgcctgctcctcctcgatgggattaggcccacagttcggaatcttggggagagccaaggaagcggtaggcacccagtaggcccacggccgtcggctgatagcaatggtgatgctgtcctacctacttgtgtaaggcattcgatcttcctcccttccatacatattgaaataaataagccgcgcaatgtgttagctattgatcagaactaaagtgaagtcagccacggggattacaaatctcggcttctcccctcatgttcctgagagtcttcccctggttttgaacacatctccctagctcgatgtcaaggtgagggattctgtcggcaacagcagtgcccttagttgcttcgtcgtaactccccgtcaccggttttattcagttaccttccagtcccactctcagagcttcctggcttgttctgctctcaaagcgggtagagctggcacacatggactctccgaaacggctgcaagatgccaagtttctcggaagaactggaagcacagagaccagaagtgccttaaggtctcgctattcagtgtggcgcttagaccggcagtggcggcagctgccctgggagcttgttagaatgtggcttctcacgcccctcctggacctacagagtcagaatctgcagttttacaggaggtccaggcttggaagttgctcgtagagacctgagacagcgcagccacgtgctggaaacaaagcatttaagtttgtgactttattttaaaaggcagcaggcagtcgacaaaccaatttcttctacttagaggcggcttcggcttctggaagtcgctaggagtataaagttgccaaccagcgctgttctcccgctgttttctgtgcacttataaatgggaagttaggtcaggatagatctctcagctattacaaggatacaaaatacgaacattctacaagttacttaacacacacacacacacacacacacacacacacacacacaaaattaattccacaggtcagtttctctgaaacattttttcactaaattctaagtcttcctggagttgcaagtgcctatctcctagacaaggcaattactcaccaactaaaatcactgtcaatctgagatttcggctgggcatgagaccatggtcaggggatgctttgaacagcctctgaggaaattagtgagtttgaaaaatggaaagatttttattactcacttggcagtaaaacctgatggggacagacgtcaggctgtttaagatcctcagaagaaaaagttgatagtgtgaatattcctaaatttgccacacgaagatgtacatgtgattataaggtgctgttgcagaagcccctgggggtgttatgggatatacactatatgggccactttaccttcctaaaatctgaaaaacttcaactactgaaacatggactgaaggttttgaatagtggatggtgaatttgaataccatcccgtgtgatttttttttctagcagactttagttttttagagcagttttaagcccacaccaaaactgagaggaagatacagcaatttctcatataccccctactaccttccagtctcc

SEQ ID NO: 5 - DNA, 2137 bp, Homo sapiens
cttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 6930 bp, Homo sapiens
gaccgttgcttggcagacactggatggttatgagcctgaacaagctgaaaaggggcaggaaaagaagtggaggcagcattcttcctatttaaagctgcatcgcttgaaaaaagttttcgcagactgtgctggagctggtgctgaaaaagggggtttgcagaggctgccctggggctggtgctgaaagaagagcccacagctgacttcatggtgctacaataacctcagaatctacttttcactctcaggagaacccacatgtctaatatttagacatgatggcaaactgggcggaagcaagacctctcctcattcttattgttttattagggcaatttgtctcaataaaagcccaggaagaagacgaggatgaaggatatggtgaagaaatagcctgcactcagaatggccagatgtacttaaacagggacatttggaaacctgccccttgtcagatctgtgtctgtgacaatggagccattctctgtgacaagatagaatgccaggatgtgctggactgtgccgaccctgtaacgccccctggggaatgctgtcctgtctgttcacaaacacctggaggtggcaatacaaattttggtagaggaagaaagggacaaaagggagaaccaggattagtgcctgttgtaacaggcatacgtggtcgtccaggaccggcaggacctccaggatcacagggaccaagaggagagcgagggccaaaaggaagacctggccctcgtggacctcagggaattgatggagaaccaggtgttcctggtcaacctggtgctccaggacctcctggacatccgtcccacccaggacccgatggcttgagcaggccgttttcagctcaaatggctgggttggatgaaaaatctggacttgggagtcaagtaggactaatgcctggctctgtgggtcctgttggcccaaggggaccacagggtttacaaggacagcaaggtggtgcaggacctacaggacctcctggtgaacctggtgatcctggaccaatgggtccgattggttcacgtggaccagagggccctcctggtaaacctggggaagatggtgaacctggcagaaatggaaatcctggtgaagtgggatttgcaggatctccgggagctcgtggatttcctggggctcctggtcttccaggtctgaagggtcaccgaggacacaaaggtcttgaaggccctaaaggtgaagttggagcacctggttccaagggtgaagctggccccactggtccaatgggtgccatgggtcctctgggtccgaggggaatgccaggagagagagggagacttgggccacagggtgctcctggacaacgaggtgcacatggtatgcctggaaaacctggaccaatgggtcctcttgggataccaggctcttctggttttccaggaaatcctggaatgaagggagaagcaggtcctacaggggcgcgaggccctgaaggtcctcaggggcagagaggtgaaactgggcccccaggtccagttggctctccaggtcttcctggtgcaataggaactgatggtactcctggtgccaaaggcccaacgggctctccgggtacctctggtcctcctggctcagcagggcctcctggatctccaggacctcagggtagcactggtcctcagggaattcgaggccaaccgggtgatccaggagttccaggtttcaaaggagaagctggcccaaaaggggaaccagggccacatggtattcagggtccgataggcccacccggtgaagaaggcaaaagaggtcccagaggtgacccaggaacagttggtcctccagggccagtgggagaaaggggtgctcctggcaatcgtggttttccaggctctgatggtttacctgggccaaagggtgctcaaggagaacggggtcctgtaggttcttcaggacccaaaggaagccagggggatccaggacgtccaggggaacctgggcttccaggtgctcggggtttgacaggaaatcctggtgttcaaggtcctgaaggaaaacttggacctttgggtgcgccaggggaagatggccgtccaggtcctccaggctccataggaatcagagggcagcccgggagcatgggccttccaggccccaaaggtagcagtggtgaccctgggaaacctggagaagcaggaaatgctggagttcctgggcagaggggagctcctggaaaagatggtgaagttggtccttctggtcctgtgggcccgccgggtctagctggtgaaagaggagaacaaggacctccaggccccacaggttttcaggggcttcctggtcctccagggcctcctggagaaggtggaaaaccaggtgatcaaggtgttcctggagatcccggagcagttggcccgttaggacctagaggagaacgaggaaatcctggggaaagaggagaacctgggataactggactccctggtgagaagggaatggctggaggacatggtcctgatggcccaaaaggcagtccaggtccatctgggacccctggagatacaggcccaccaggtcttcaaggtatgccgggagaaagaggaattgcaggaactcctggccccaagggtgacagaggtggcataggagaaaaaggtgctgaaggcacagctggaaatgatggtgcaagaggtcttccaggtcctttgggccctccaggtccggcaggtcctactggagaaaagggtgaacctggtcctcgaggtttagttggccctcctggctcccggggcaatcctggttctcgaggtgaaaatgggccaactggagctgttggttttgccggaccccagggtcctgacggacagcctggagtaaaaggtgaacctggagagccaggacagaagggagatgctggttctcctggaccacaaggtttagcaggatcccctggccctcatggtcctaatggtgttcctggactaaaaggtggtcgaggaacccaaggtccgcctggtgctacaggatttcctggttctgcgggcagagttggacctccaggccctgctggagctccaggacctgcgggacccctaggggaacccgggaaggagggacctccaggtcttcgtggggaccctggctctcatgggcgtgtgggagatcgaggaccagctggcccccctggtggcccaggagacaaaggggacccaggagaagatgggcaacctggtccagatggcccccctggtccagctggaacgaccgggcagagaggaattgttggcatgcctgggcaacgtggagagagaggcatgcccggcctaccaggcccagcgggaacaccaggaaaagtaggaccaactggtgcaacaggagataaaggtccacctggacctgtggggcccccaggctccaatggtcctgtaggggaacctggaccagaaggtccagctggcaatgatggtaccccaggacgggatggtgctgttggagaacgtggtgatcgtggagaccctgggcctgcaggtctgccaggctctcagggtgcccctggaactcctggccctgtgggtgctccaggagatgcaggacaaagaggagatccgggttctcggggtcctataggaccacctggtcgagctgggaaacgtggattacctggaccccaaggacctcgtggtgacaaaggtgatcatggagaccgaggcgacagaggtcagaagggccacagaggctttactggtcttcagggtcttcctggccctcctggtccaaatggtgaacaaggaagtgctggaatccctggaccatttggcccaagaggtcctccaggcccagttggtccttcaggtaaagaaggaaaccctgggccacttgggccaattggacctccaggtgtacgaggcagtgtaggagaagcaggacctgagggccctcctggtgagcctggcccacctggccctccgggtccccctggccaccttacagctgctcttggggatatcatggggcactatgatgaaagcatgccagatccacttcctgagtttactgaagatcaggcggctcctgatgacaaaaacaaaacggacccaggggttcatgctaccctgaagtcactcagtagtcagattgaaaccatgcgcagccccgatggctcgaaaaagcacccagcccgcacgtgtgatgacctaaagctttgccattccgcaaagcagagtggtgaatactggattgatcctaaccaaggatctgttgaagatgcaatcaaagtttactgcaacatggaaacaggagaaacatgtatttcagcaaacccatccagtgtaccacgtaaaacctggtgggccagtaaatctcctgacaataaacctgtttggtatggtcttgatatgaacagagggtctcagttcgcttatggagaccaccaatcacctaatacagccattactcagatgacttttttgcgccttttatcaaaagaagcctcccagaacatcacttacatctgtaaaaacagtgtaggatacatggacgatcaagctaagaacctcaaaaaagctgtggttctcaaaggggcaaatgacttagatatcaaagcagagggaaatattagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaaagtaagccaagacacatcgacaatgagcaccaccatcaatgaccaccgccattcacaagaactttgactgtttgaagttgatcctgagactcttgaagtaatggctgatcctgcatcagcattgtatatatggtcttaagtgcctggcctccttatccttcagaatatttattttacttacaatcctcaagttttaattgattttaaatatttttcaatacaacagtttaggtttaagatgaccaatgacaatgaccacctttgcagaaagtaaactgattgaataaataaatctccgttttcttcaatttatttcagtgtaatgaaaaagttgcttagtatttatgaggaaattcttcttcctggcaggtagcttaaagagtggggtatatagagccacaacacatgtttattttgcttggctgcagttgaaaaatagaaattagtgcccttttgtgacctctcattccaagattgtcaattaaaaatgagtttaaaatgtttaacttgtgatcgagacctacatgcatgtcttgatattgtgtaactataatagagactctttaaggagaatcttaaaaaaaaaaaaacgtttctcactgtcttaaatagaatttttaaatagtatatattcagtggcattttggagaacaaagtgaatttacttcgacttcttaaatttttgtaaaagactataagtttagacatctttctcattcaaatttaaagatatctttctcctcttgatcaatctatcaatattgatagaagtcacactagtatataccatttaatacatttacactttcttatttaagaagatattgaatgcaaaataattgacatatagaactttacaaacatatgtccaaggactctaaattgagactcttccacatgtacaatctcatcatcctgaagcctataatgaagaaaaagatctagaaactgagttgtggagctgactctaatcaaatgtgatgattggaattagaccatttggcctttgaactttcataggaaaaatgacccaacatttcttagcatgagctacctcatctctagaagctgggatggacttactattcttgtttatattttagatactgaaaggtgctatgcttctgttattattccaagactggagataggcagggctaaaaaggtattattatttttcctttaatgatggtgctaaaattcttcctataaaattccttaaaaataaagatggtttaatcactaccattgtgaaaacataactgttagacttcccgtttctgaaagaaagagcatcgttccaatgcttgttcactgttcctctgtcatactgtatctggaatgctttgtaatacttgcatgcttcttagaccagaacatgtaggtccccttgtgtctcaatactttttttttcttaattgcatttgttggctctattttaatttttttcttttaaaataaacagctgggaccatcccaaaagacaagccatgcatacaactttggtcatgtatctctgcaaagcatcaaattaaatgcacgcttttgtcatgtcagtggtttttgttttgtgaaattcctttgaccatattagatctatttcatttccaatagtgaaaaggagatgtggtggtatactttgtttgccatttgtttaaaagatacaacggataccttctatcatgtatgtactggcttataaatgaaaatctatctacaacattacccacaaaggcaacatgacaccaattatcactgcctctgcccttaaaaatgtcagagtagtattattgataaaaagggcaagcaatagatttttcatgactgaataaactgtaataataaaacatatgtctcaaagtgtatcacatatgaatttagcctaattgttttcagtttcattctcaatatttagtttacaacatcattttcccctaaactggttatattttgacctgtatatcttaaatttgagtatttatatgcctaaatacatgtgtgagttttgtttgacttccaagtccaaactataagattatataagttcatatagatgaatcagaaatatgtggtaatactattaagtcacaaacactaacaatttccaactatagaaataacagttcttatttggattttgggaatgctaccaata

SEQ ID NO: 7 - DNA, 510 bp, Homo sapiens
tgaggctgccttataaagcaccaagaggctgccagtgggacattttctcggccctgccagcccccaggaggaaggtgggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggagatgtcacagattcctggcagagccatggtcccaggcttcccaaaagtgtttgttggcaattattcccctaggctgagcctgctcatgtacctctgattaataaatgcttatgaaatga

SEQ ID NO: 8 - DNA, 2013 bp, Homo sapiens
accccgtccagcttcatccgcagaggagcctcggccaggcttgccagggcgcccccagcccctccccaggccgcgagcgcccctgccgcggtgcctggcctccccgcccagactgcagggacagcacccggtaactgcgagtggagcggaggacccgagcggctgaggagagaggaggcggcggcttagctgctacggggtccggccggcgccctcccgaggggggctcaggaggaggaaggaggacccgtgcgagaatgcctctgccctggagccttgcgctcccgctgctgctctcctgggtggcaggtggtttcgggaacgcggccagtgcaaggcatcacgggttgttagcatcggcacgtcagcctggggtctgtcactatggaactaaactggcctgctgctacggctggagaagaaacagcaagggagtctgtgaagctacatgcgaacctggatgtaagtttggtgagtgcgtgggaccaaacaaatgcagatgctttccaggatacaccgggaaaacctgcagtcaagatgtgaatgagtgtggaatgaaaccccggccatgccaacacagatgtgtgaatacacacggaagctacaagtgcttttgcctcagtggccacatgctcatgccagatgctacgtgtgtgaactctaggacatgtgccatgataaactgtcagtacagctgtgaagacacagaagaagggccacagtgcctgtgtccatcctcaggactccgcctggccccaaatggaagagactgtctagatattgatgaatgtgcctctggtaaagtcatctgtccctacaatcgaagatgtgtgaacacatttggaagctactactgcaaatgtcacattggtttcgaactgcaatatatcagtggacgatatgactgtatagatataaatgaagagaaaatgaaagaggggcttgaggatgagaaaagagaagagaaagccctgaagaatgacatagaggagcgaagcctgcgaggagatgtgtttttccctaaggtgaatgaagcaggtgaattcggcctgattctggtccaaaggaaagcgctaacttccaaactggaacataaagcagatttaaatatctcggttgactgcagcttcaatcatgggatctgtgactggaaacaggatagagaagatgattttgactggaatcctgctgatcgagataatgctattggcttctatatggcagttccggccttggcaggtcacaagaaagacattggccgattgaaacttctcctacctgacctgcaaccccaaagcaacttctgtttgctctttgattaccggctggccggagacaaagtcgggaaacttcgagtgtttgtgaaaaacagtaacaatgccctggcatgggagaagaccacgagtgaggatgaaaagtggaagacagggaaaattcagttgtatcaaggaactgatgctaccaaaagcatcatttttgaagcagaacgtggcaagggcaaaaccggcgaaatcgcagtggatggcgtcttgcttgtttcaggcttatgtccagatagccttttatctgtggatgactgaatgttactatctttatatttgactttgtatgtcagttccctggtttttttgatattgcatcataggacctctggcattttagaattactagctgaaaaattgtaatgtaccaacagaaatattattgtaagatgcctttcttgtataagatatgccaatatttgctttaaatatcatatcactgtatcttctcagtcatttctgaatctttccacattatattataaaatatggaaatgtcagtttatctcccctcctcagtatatctgatttgtataagtaagttgatgagcttctctctacaacatttctagaaaatagaaaaaaaagcacagagaaatgtttaactgtttgactcttatgatacttcttggaaactatgacatcaaagatagacttttgcctaagtggcttagctgggtctttcatagcc

SEQ ID NO: 9 - DNA, 1236 bp, Homo sapiens
ctgcggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaaaaaaaaaaaaaaaaa

SEQ ID NO: 10 - DNA, 20 bp, Artificial Sequence
  AZGP1 forward primer
ctctgcggaaatacctgaaa

SEQ ID NO: 11 - DNA, 20 bp, Artificial Sequence
  AZGP1 reverse primer
tgaagaacatctccccgtaa

SEQ ID NO: 12 - DNA, 18 bp, Artificial Sequence
  CXCL3 forward primer
ggtgctccccttgttcag

SEQ ID NO: 13 - DNA, 18 bp, Artificial Sequence
  CXCL3 reverse primer
agggaattcacctcaaga

SEQ ID NO: 14 - DNA, 18 bp, Artificial Sequence
  CXCL6 forward primer
agatccctggacccagta

SEQ ID NO: 15 - DNA, 18 bp, Artificial Sequence
  CXCL6 reverse primer
ttgccaaagggttcaata

SEQ ID NO: 16 - DNA, 18 bp, Artificial Sequence
  AGT forward primer
gctgcaaaacttgacacc

SEQ ID NO: 17 - DNA, 18 bp, Artificial Sequence
  AGT reverse primer
attgcctgtagcctgtca

SEQ ID NO: 18 - DNA, 20 bp, Artificial Sequence
  FCGR3A forward primer
gcttgttgggagtaaaaatg

SEQ ID NO: 19 - DNA, 18 bp, Artificial Sequence
  FCGR3A reverse primer
tccagtcttgttgagctt

SEQ ID NO: 20 - DNA, 20 bp, Artificial Sequence
  Col5A2 forward primer
gacctcgtggtgacaaaggt

SEQ ID NO: 21 - DNA, 20 bp, Artificial Sequence
  Col5A2 reverse primer
agccgcctgatcttcagtaa

SEQ ID NO: 22 - DNA, 19 bp, Artificial Sequence
  S100P forward primer
agacagccatgggcatgat

SEQ ID NO: 23 - DNA, 21 bp, Artificial Sequence
  S100P reverse primer
tcatttgagtcctgccttctc

SEQ ID NO: 24 - DNA, 20 bp, Artificial Sequence
  EGFL6 forward primer
gcatgaaaaagaaggcaaaa

SEQ ID NO: 25 - DNA, 20 bp, Artificial Sequence
  EGFL6 reverse primer
tgtcattcttcagggctttc

SEQ ID NO: 26 - DNA, 21 bp, Artificial Sequence
  CTHRC1 forward primer
tcatcgcacttcttctgtgga

SEQ ID NO: 27 - DNA, 21 bp, Artificial Sequence
  CTHRC1 reverse primer
gccaacccagatagcaacatc

SEQ ID NO: 28 - DNA, 20 bp, Artificial Sequence
  beta-actin forward primer
gatcattgctcctcctgagc

SEQ ID NO: 29 - DNA, 20 bp, Artificial Sequence
  beta-actin reverse primer
actcctgcttgctgatccac